(19) United States
(12) Patent Application Publication
(10) Pub. No.: US 2010/0279996 A1
(43) Pub. Date: Nov. 4, 2010

(54) NOVEL ANALGESIC THAT BINDS FILAMIN A

(76) Inventors: Lindsay Burns Barbier, Palo Alto, CA (US); Hoau-Yan Wang, Philadelphia, PA (US); Nan-Horng Lin, Vernon Hills, IL (US); Andrei Blasko, San Bruno, CA (US)

Correspondence Address: Husch Blackwell Sanders, LLP;Husch Blackwell Sanders LLP Welsh & Katz, 120 S RIVERSIDE PLAZA, 22ND FLOOR, CHICAGO, IL, 60606, US

(21) Appl. No.: 12/435,284

(22) Filed: May 4, 2009

(51) Int. Cl. A61K 31/397; C07D 265/32; A61K 31/5375; C07D 279/12; A61K 31/54; C07D 243/08; A61K 31/551; C07D 223/04; A61K 31/55; C07D 413/02; A61K 31/5377; A61P 29/00

(52) U.S. Cl. 514/210.01; 544/174; 514/239.2; 544/59; 514/227.5; 540/575; 514/218; 540/609; 514/212.01; 544/121; 514/235.8; 540/450

(57) ABSTRACT

A compound, composition and method are disclosed that can provide analgesia. A contemplated compound has a structure that corresponds to Formula I, wherein R¹ and R² are substituents, and W is a ring structure that are defined within.

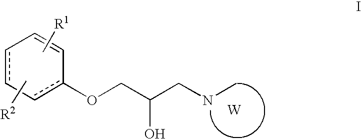

### CROSS-REFERENCE TO RELATED APPLICATION

This applications claims priority from application Ser. No. 12/263,257 that was filed on Oct. 31, 2008, and whose disclosures are incorporated herein by reference.

### TECHNICAL FIELD

This invention contemplates a composition and related method for providing opioid-strength analgesia while minimizing analgesic tolerance, physical dependence and addiction. More particularly, a composition and method are described that utilize a small molecule to inhibit the interaction of the mu opioid receptor with filamin A, either by binding to filamin A itself or by mimicking filamin A′ s binding to the mu opioid receptor. Preferably, the composition prevents this mu opioid receptor-filamin A interaction and also functions as a mu opioid receptor agonist. Most preferably, the composition binds filamin A with picomolar or sub-picomolar affinity.

### BACKGROUND OF THE INVENTION

Opiates are powerful analgesics (agents used for the treatment of pain), but their use is hampered by non-trivial side effects, tolerance to the analgesic effects, physical dependence resulting in withdrawal effects, and by concerns surrounding the possibility of addiction.

Opiates produce analgesia by activation of opioid receptors that belong to the rhodopsin-like superfamily of G protein-coupled receptors (GPCRs). Adaptive responses of opioid receptors contribute to the development of analgesic tolerance and physical dependence, and possibly also to components of opioid addiction.

Opiates produce analgesia by activation of mu (μ) opioid receptor-linked inhibitory G protein signaling cascades and related ion channel interactions that suppress cellular activities by hyperpolarization. The μ opioid receptor (MOR) preferentially couples to pertussis toxin-sensitive G proteins, Gαi/o (inhibitory/other), and inhibits the adenylyl cyclase/cAMP pathway (Laugwitz et al., 1993 Neuron 10:233-242; Connor et al., 1999 Clin Exp Pharmacol Physiol 26:493-499). The analgesic effects of MOR activation have been predominantly attributed to the Gβγ dimer released from the Gαi/o protein, which activates G protein activated inwardly rectifying potassium (GIRK) channels (Ikeda et al., 2000 Neurosci Res 38:113-116) and inhibits voltage-dependent calcium channels (VDCCs) (Saegusa et al., 2000 Proc Natl Acad Sci USA 97:6132-6137), thereby suppressing cellular activities by hyperpolarization.

Adenylyl cyclase inhibition can also contribute to opioid analgesia, or importantly, its activation can contribute to analgesic tolerance. The role of adenylyl cyclase inhibition or activation in opioid analgesia and analgesic tolerance, respectively, is evidenced by overexpression of adenylyl cyclase type 7 in the CNS of mice leading to more rapid tolerance to morphine (Yoshimura et al., 2000 Mol Pharmacol 58:1011-1016). Additionally, adenylyl cyclase activation has been suggested to elicit analgesic tolerance or tolerance-associated hyperalgesia (Wang et al., 1997 J Neurochem 68:248-254). Although the superactivation of adenylyl cyclase after chronic opioid administration is more often viewed as a hallmark of opioid dependence than as a mediator of tolerance (Nestler, 2001 Am J Addict 10:201-217), both are consequences of chronic opioid administration, and tolerance often worsens dependence. Chronic pain patients who have escalated their opioid dose over time often experience more withdrawal than patients on a constant dose.

An important but underemphasized cellular consequence of chronic opioid treatment is MOR excitatory signaling, by activation of adenylyl cyclase, in place of the usual inhibitory signaling or inhibition of adenylyl cyclase (Crain et al., 1992 Brain Res 575:13-24; Crain et al., 2000 Pain 84:121-131; Gintzler et al., 2001 Mol Neurobiol 21:21-33; Wang et al., 2005 Neuroscience 135:247-261). This change in signaling is caused by a switch in G protein coupling from Gi/o to Gs (Wang et al., 2005 Neuroscience 135:247-261) and may be a result of the decreased efficiency of coupling to the native G proteins, the usual index of desensitization (Sim et al., 1996 J Neurosci 16:2684-2692) and still commonly considered the reason for analgesic tolerance.

The chronic opioid-induced MOR-G protein coupling switch (Wang et al 2005 Neuroscience 135:247-261; Chakrabarti et al., 2005 Mol Brain Res 135:217-224) is accompanied by stimulation of adenylyl cyclase II and IV by MOR-associated Gβγ dimers (Chakrabarti et al., 1998 Mol Pharmacol 54:655-662; Wang et al., 2005 Neuroscience 135:247-261). The interaction of the Gβγ dimer with adenylyl cyclase had previously been postulated to be the sole signaling change underlying the excitatory effects of opiates (Gintzler et al., 2001 Mol Neurobiol 21:21-33). It has further been shown that the Gβγ that interacts with adenylyl cyclases originates from the Gs protein coupling to MOR and not from the Gi/o proteins native to MOR (Wang et al., 2006 J Neurobiol 66:1302-1310).

Thus, MORs are normally inhibitory G protein-coupled receptors that couple to Gi or Go proteins to inhibit adenylyl cyclase and decrease production of the second messenger cAMP, as well as to suppress cellular activities via ion channel-mediated hyperpolarization. Opioid analgesic tolerance and dependence are also associated with that switch in G protein coupling by MOR from Gi/o to Gs (Wang et al., 2005 Neuroscience 135:247-261). This switch results in activation of adenylyl cyclase that provides essentially opposite, stimulatory, effects on the cell.

Controlling this switch in G protein coupling by MOR is the scaffolding protein filamin A, and compounds that bind a particular segment of filamin A with high affinity, like naloxone (NLX) and naltrexone (NTX), can prevent this switch (Wang et al, 2008 PLoS One 3:e1554) and the associated analgesic tolerance and dependence (Wang et al., 2005 Neuroscience 135:247-261). This switch in G protein coupling also occurs acutely, though transiently, and is potentially linked to the acute rewarding or addictive effects of opioid drugs, through CREB activation as a result of increased cAMP accumulation (Wang et al., 2009 PLoS ONE 4(1):e4282).

Ultra-low-dose NLX or NTX have been shown to enhance opioid analgesia, minimize opioid tolerance and dependence (Crain et al., 1995 Proc Natl Acad Sci USA 92:10540-10544; Powell et al. 2002. JPET 300:588-596), as well as to attenuate the addictive properties of opioids (Leri et al., 2005 Pharmacol Biochem Behav 82:252-262; Olmstead et al., 2005 Psychopharmacology 181:576-581). An ultra-low dose of opioid antagonist was an amount initially based on in vitro studies of nociceptive dorsal root ganglion neurons and on in vivo mouse studies, wherein the amount of the excitatory opioid receptor antagonist administered is about 1000- to about 10,000,000-fold less, preferably about 10,000- to about 1,000,000-fold less than the amount of opioid agonist administered. It has long been hypothesized that ultra-low-dose opioid antagonists enhance analgesia and alleviate tolerance/dependence by blocking the excitatory signaling opioid receptors that underlie opioid tolerance and hyperalgesia (Crain et al., 2000 Pain 84:121-131). Later research has shown that the attenuation of opioid analgesic tolerance, dependence and addictive properties by ultra-low-dose, defined herein, naloxone or naltrexone, occurs by preventing the MOR-Gs coupling that results from chronic opiate administration (Wang et al., 2005 Neuroscience 135:247-261), and that the prevention of MOR-Gs coupling is a result of NLX or NTX binding to filamin A at approximately 4 picomolar affinity (Wang et al, 2008 PLoS One 3:e1554).

Found in all cells of the brain, CREB is a transcription factor implicated in addiction as well as learning and memory and several other experience-dependent, adaptive (or maladaptive) behaviors (Carlezon et al., 2005 Trends Neurosci 28:436-445). In general, CREB is inhibited by acute opioid treatment, an effect that is completely attenuated by chronic opioid treatment, and activated during opioid withdrawal (Guitart et al., 1992 J Neurochem 58:1168-1171). However, a regional mapping study showed that opioid withdrawal activates CREB in locus coeruleus, nucleus accumbens and amygdala but inhibits CREB in lateral ventral tegemental area and dorsal raphe nucleus (Shaw-Luthman et al., 2002 J Neurosci 22:3663-3672).

In the striatum, CREB activation has been viewed as a homeostatic adaptation, attenuating the acute rewarding effects of drugs (Nestler, 2001 Am J Addict 10:201-217; Nestler, 2004 Neuropharmacology 47:24-32). This view is supported by nucleus accumbens overexpression of CREB or a dominant-negative mutant respectively reducing or increasing the rewarding effects of opioids in the conditioned place preference test (Barot et al., 2002 Proc Natl Acad Sci USA 99:11435-11440). In conflict with this view, however, reducing nucleus accumbens CREB via antisense attenuated cocaine reinforcement as assessed in self-administration (Choi et al., 2006 Neuroscience 137:373-383). Clearly, CREB activation is implicated in addiction, but whether it directly contributes to the acute rewarding effects of drugs or initiates a homeostatic regulation thereof appears less clear.

The several-fold increase in pS¹³³CREB reported by Wang et al., 2009 PLoS ONE 4(1):e4282 following acute, high-dose morphine may indicate acute dependence rather than acute rewarding effects. However, the transient nature of the MOR-Gs coupling correlating with this CREB activation suggests otherwise. In fact, the correlation of pS¹³³CREB with the Gs coupling by MOR following this acute high-dose morphine exposure, as well as the similar treatment effects on both, suggest that this alternative signaling mode of MOR can contribute to the acute rewarding or addictive effects of opioids. This counterintuitive notion can explain the apparent paradox that ultra-low-dose NTX, while enhancing the analgesic effects of opioids, decreases the acute rewarding or addictive properties of morphine or oxycodone as measured in conditioned place preference or self-administration and reinstatement paradigms.

In considering analgesic tolerance, opioid dependence, and opioid addiction together as adaptive regulations to continued opioid exposure, a treatment that prevents MOR's signaling adaptation of switching its G protein partner can logically attenuate these seemingly divergent behavioral consequences of chronic opioid exposure. However, the acute rewarding effects of opioids are not completely blocked by ultra-low-dose opioid antagonists, suggesting that a MOR-Gs coupling can only partially contribute to the addictive or rewarding effects.

Even though ultra-low-dose NTX blocks the conditioned place preference to oxycodone or morphine (Olmstead et al., 2005 Psychopharmacology 181:576-581), its co-self-administration only reduces the rewarding potency of these opioids but does not abolish self-administration outright (Leri et al., 2005 Pharmacol Biochem Behav 82:252-262). Nevertheless, it is possible that a direct stimulatory effect on VTA neurons, as opposed to the proposed disinhibition via inhibition of GABA interneurons (Spanagel et al., 1993 Proc Natl Acad Sci USA 89:2046-2050), can play some role in opioid reward. Finally, a MOR-Gs coupling mediation of reward, increasing with increasing drug exposure, is in keeping with current theories that the escalation of drug use signifying drug dependence can not indicate a “tolerance” to rewarding effects but instead a sensitization to rewarding effects (Zernig et al., 2007 Pharmacology 80:65-119).

The above results reported in Wang et al., 2009 PLoS ONE 4(1):e4282 demonstrated that acute, high-dose morphine causes an immediate but transient switch in G protein coupling by MOR from Go to Gs similar to the persistent switch caused by chronic morphine. Ultra-low-dose NLX or NTX prevented this switch and attenuated the chronic morphine-induced coupling switch by MOR. The transient nature of this acute altered coupling suggests the receptor eventually recovers and couples to its native G protein.

With chronic opioid exposure, the receptor can lose the ability to recover and continue to couple to Gs, activating the adenylyl cyclase/cAMP pathway, upregulating protein kinase A, and phosphorylating CREB as one downstream effector example. The persistently elevated phosphorylated CREB can then shape the expression of responsive genes including those closely related to drug addiction and tolerance. Importantly, the equivalent blockade of Gs coupling and pS¹³³CREB by the pentapeptide binding site of naloxone (NLX) and naltrexone (NTX) on FLNA further elucidates the mechanism of action of ultra-low-dose NLX and NTX in their varied effects.

These data further strengthen a mechanistic basis for MOR-Gs coupling through the interaction between FLNA and MOR and that disrupting this interaction, either by NLX/NTX binding to FLNA or via a FLNA peptide decoy for MOR, the altered coupling is prevented, resulting in attenuation of tolerance, dependence and addictive properties associated with opioid drugs.

The combination of ultra-low-dose opioid antagonists with opioid agonists formulated together in one medication has been shown to alleviate many of these undesirable aspects of opioid therapy (Burns, 2005 Recent Developments in Pain Research 115-136, ISBN:81-308-0012-8). This approach shows promise for an improvement in analgesic efficacy, and animal data suggests reduced addictive potential. The identification of the cellular target of ultra-low-dose NLX or NTX in their inhibition of mu opioid receptor-Gs coupling as a pentapeptide segment of filamin A (Wang et al., 2008 PLoS ONE 3(2):e1554) has led to development of assays to screen against this target to create a new generation of pain therapeutics that can provide long-lasting analgesia with minimal tolerance, dependence and addictive properties. Importantly, the non-opioid cellular target of ultra-low-dose NLX or NTX, FLNA, provides potential for developing either a therapeutic combination of which one component is not required to be ultra-low-dose, or a single-entity novel analgesic. The present invention identifies a compound that is similar to or more active than DAMGO in activating the mu (μ) opioid receptor (MOR), and that also binds to filamin A (FLNA; the high-affinity binding site of naloxone [NLX] and naltrexone [NTX]), thereby preventing the Gi/o-to-Gs coupling switch of MOR to attenuate opioid tolerance, dependence and addiction.

### BRIEF SUMMARY OF THE INVENTION

The present invention contemplates an analgesic compound and a method of reducing pain in a host mammal in need thereof by administering a composition containing such a compound. A compound contemplated by the present invention corresponds in structure to Formula I

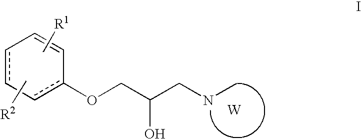

In a compound of Formula I, R¹ and R² are the same or different and are independently H, halogen, C₁-C₆ hydrocarbyl, C₁-C₆ acyl, C₁-C₆ hydrocarbyloxy and NR³R⁴ wherein R³ and R⁴ are the same or different and are H, C₁-C₄ hydrocarbyl, C₁-C₄ acyl, C₁-C₄ hydrocarbylsulfonyl, or R³ and R⁴ together with the depicted nitrogen form a 5-7-membered ring that optionally contains 1 or 2 additional hetero atoms that independently are nitrogen, oxygen or sulfur. W is a ring structure that contains 5 to 8 atoms in the ring including the depicted nitrogen. W and can optionally contain: a) 1 or 2 further hetero atoms that are independently oxygen, nitrogen or sulfur, and b) one or more substituent groups bonded to one or more ring atoms, in which the one or more substituents contain a total of up to 12 atoms selected from the group consisting of carbon, nitrogen, oxygen and sulfur, and mixtures thereof. A dotted line ( [symbol] ) represents an optional double bond. In regard to a contemplated compound, R¹ and R² are other than methyl and isopropyl, respectively, when W is N-morpholinyl or dimethyl-N-morpholinyl and the optional double bonds are absent.

In one preferred embodiment, a contemplated compound corresponds in structure to Formula IA

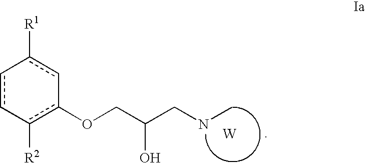

Here, R¹ and R² are the same or different and are independently H, or C₁-C₆ hydrocarbyl; W is a ring structure that contains 5 to 8 atoms in the ring including the depicted nitrogen, and can optionally contain: a) 1 or 2 further hetero atoms that are independently oxygen, nitrogen or sulfur, and b) one or more substituent groups bonded to one or more ring atoms, in which the one or more substituent contain a total of up to 12 atoms selected from the group consisting of carbon, nitrogen, oxygen and sulfur, and mixtures thereof. The dotted line ( [symbol] ) represents 1, 2, or 3 optional double bonds, and R¹ and R² are other than methyl and isopropyl, respectively, when W is N-morpholinyl or dimethyl-N-morpholinyl, and the optional double bonds are absent.

In preferred practice for a compound of either Formula I or Formula Ia, W further includes one or more substituent groups bonded to one or more ring atoms, in which those one or more substituents contain a total of up to 12 atoms selected from the group consisting of carbon, nitrogen, oxygen and sulfur, and mixtures thereof. In one preferred embodiment, a compound of Formulas I and Ia has the structure of Formula II, whereas in another preferred embodiment, a compound of Formulas I and Ia has the structure of a compound of Formula III.

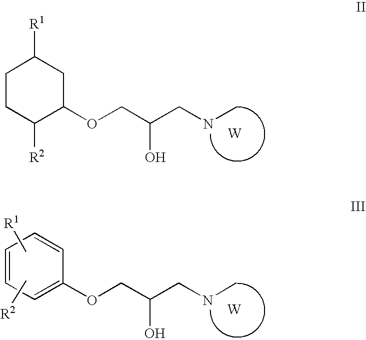

W, in a compound of both Formulas II and III, is as previously defined for a compound of Formulas I and Ia. R¹ and R² for a compound of Formula II are defined as are R¹ and R² for a compound of Formula Ia, whereas R¹ and R² for a compound of Formula III are defined as are R¹ and R² for a compound of Formula I.

More preferably, the R¹ and R² of a compound of Formula II contain 3 to 5 carbon atoms. For some compounds of Formula III, R¹ is H and R² is halogen, C₁-C₆ hydrocarbyl, C₁-C₆ acyl, C₁-C₆ hydrocarbyloxy or NR³R⁴, whereas for others, both R groups are other than H. In a compound of either formula, W can optionally contain 1 or 2 further hetero atoms that are independently oxygen, nitrogen or sulfur, and more preferably still contains at least one such hetero atom. It is also preferred that W further includes one or more substituent groups bonded to one or more ring atoms, in which the one or more substituents contain a total of up to 12 atoms selected from the group consisting of carbon, nitrogen, oxygen and sulfur, and mixtures thereof.

A contemplated method comprises administering to a host mammal host in need thereof a pharmaceutical composition containing an analgesia effective amount of a compound of Formula I dissolved or dispersed in a physiologically tolerable carrier. For a contemplated method, R¹ and R² can be methyl and isopropyl, respectively, when W is N-morpholinyl or dimethyl-N-morpholinyl and the optional double bonds are absent. A contemplated composition is typically administered a plurality of times over a period of days, and is preferably administered a plurality of times in one day.

The present invention has several benefits and advantages.

One benefit is that analgesia can be provided at morphine-like potency by a compound that does not have a narcotic structure.

An advantage of the invention is that analgesia can be provided by administration of a contemplated composition either perorally or parenterally.

A further benefit of the invention is that as indicated by the initial data, a contemplated compound provides the analgesic effects characteristic of opioid drugs but does not cause analgesic tolerance or dependence.

Another advantage of the invention as also indicated by the initial data is that a contemplated compound provides the analgesic effects and does not have the addictive potential of opioid drugs.

Still further benefits and advantages will be apparent to a skilled worker from the description that follows.

### Abbreviations and Short Forms

The following abbreviations and short forms are used in this specification.

“MOR” means μ-opioid receptor

“FLNA” means filamin A

“NLX” means naloxone

“NTX” means naltrexone

“Gαi/o” means G protein alpha subunit-inhibitory/other conformation, inhibits adenylyl cyclase

“Gαs” means G protein alpha subunit-stimulatory conformation stimulates adenylyl cyclase

“Gβγ” means G protein beta gamma subunit

“cAMP” means cyclic adenosine monophosphate

“CREB” means cAMP Response Element Binding protein

“IgG” means Immunoglobulin G

### DEFINITIONS

In the context of the present invention and the associated claims, the following terms have the following meanings:

The articles “a” and “an” are used herein to refer to one or to more than one (i.e., to at least one) of the grammatical object of the article. By way of example, “an element” means one element or more than one element.

As used herein, the term “hydrocarbyl” is a short hand term to include straight and branched chain aliphatic as well as alicyclic groups or radicals that contain only carbon and hydrogen. Thus, alkyl, alkenyl and alkynyl groups are contemplated, whereas aromatic hydrocarbons such as phenyl and naphthyl groups, which strictly speaking are also hydrocarbyl groups, are referred to herein as aryl groups, substituents, moieties or radicals, as discussed hereinafter. An aralkyl group such as benzyl or phenethyl is deemed a hydrocarbyl group. Where a specific aliphatic hydrocarbyl substituent group is intended, that group is recited; i.e., C₁-C₄ alkyl, methyl or dodecenyl. Exemplary hydrocarbyl groups contain a chain of 1 to about 12 carbon atoms, and preferably one to about 7 carbon atoms, and preferably 1 to about 7 carbon atoms, and more preferably 1 to 4 carbon atoms of an alkyl group.

A particularly preferred hydrocarbyl group is an alkyl group. As a consequence, a generalized, but more preferred substituent can be recited by replacing the descriptor “hydrocarbyl” with “alkyl” in any of the substituent groups enumerated herein.

Examples of alkyl radicals include methyl, ethyl, n-propyl, isopropyl, n-butyl, isobutyl, sec-butyl, tert-butyl, pentyl, iso-amyl, hexyl, octyl, decyl, dodecyl and the like. Examples of suitable alkenyl radicals include ethenyl (vinyl), 2-propenyl, 3-propenyl, 1,4-pentadienyl, 1,4-butadienyl, 1-butenyl, 2-butenyl, 3-butenyl, decenyl and the like. Examples of alkynyl radicals include ethynyl, 2-propynyl, 3-propynyl, decynyl, 1-butynyl, 2-butynyl, 3-butynyl, and the like.

Usual chemical suffix nomenclature is followed when using the word “hydrocarbyl” except that the usual practice of removing the terminal “yl” and adding an appropriate suffix is not always followed because of the possible similarity of a resulting name to one or more substituents. Thus, a hydrocarbyl ether is referred to as a “hydrocarbyloxy” group rather than a “hydrocarboxy” group as may possibly be more proper when following the usual rules of chemical nomenclature. Illustrative hydrocarbyloxy groups include methoxy, ethoxy, and cyclohexenyloxy groups. On the other hand, a hydrocarbyl group containing a —C(O)O— functionality is referred to as a hydrocarboyl (acyl) or hydrocarboyloxy group inasmuch as there is no ambiguity. Exemplary hydrocarboyl and hydrocarboyloxy groups include acyl and acyloxy groups, respectively, such as acetyl and acetoxy, acryloyl and acryloyloxy.

A “carboxyl” substituent is a —C(O)OH group. A C₁-C₆ hydrocarbyl carboxylate is a C₁-C₆ hydrocarbyl ester of a carboxyl group. A carboxamide is a —C(O)NR³R⁴ substituent, where the R groups are defined elsewhere. Illustrative R³ and R⁴ groups that together with the depicted nitrogen of a carboxamide form a 5-7-membered ring that optionally contains 1 or 2 additional hetero atoms that independently are nitrogen, oxygen or sulfur, include morpholinyl, piprazinyl, oxathiazolyl, 1,2,3-triazolyl, 1,2,4-triazolyl, pyrazolyl, 1,2,4-oxadiazinyl and azepinyl groups.

As a skilled worker will understand, a substituent that cannot exist such as a C₁ alkenyl or alkynyl group is not intended to be encompassed by the word “hydrocarbyl”, although such substituents with two or more carbon atoms are intended.

The term “aryl”, alone or in combination, means a phenyl or naphthyl radical that optionally carries one or more substituents selected from hydrocarbyl, hydrocarbyloxy, halogen, hydroxy, amino, nitro and the like, such as phenyl, p-tolyl, 4-methoxyphenyl, 4-(tert-butoxy)phenyl, 4-fluorophenyl, 4-chlorophenyl, 4-hydroxyphenyl, and the like. The term “arylhydrocarbyl”, alone or in combination, means a hydrocarbyl radical as defined above in which one hydrogen atom is replaced by an aryl radical as defined above, such as benzyl, 2-phenylethyl and the like. The term “arylhydrocarbyloxycarbonyl”, alone or in combination, means a radical of the formula —C(O)—O— arylhydrocarbyl in which the term “arylhydrocarbyl” has the significance given above. An example of an arylhydrocarbyloxycarbonyl radical is benzyloxycarbonyl. The term “aryloxy” means a radical of the formula aryl-O— in which the term aryl has the significance given above. The term “aromatic ring” in combinations such as substituted-aromatic ring sulfonamide, substituted-aromatic ring sulfinamide or substituted-aromatic ring sulfonamide means aryl or heteroaryl as defined above.

As used herein, the term “binds” refers to the adherence of molecules to one another, such as, but not limited to, peptides or small molecules such as the compounds disclosed herein, and opioid antagonists, such as naloxone or naltrexone.

As used herein, the term “selectively binds” refers to binding as a distinct activity. Examples of such distinct activities include the independent binding to filamin A or a filamin A binding peptide, and the binding of a compound discussed above to a μ opioid receptor.

As used herein, the term “FLNA-binding compound” refers to a compound that binds to the scaffolding protein filamin A, or more preferably to a polypeptide comprising residues-Val-Ala-Lys-Gly-Leu-(SEQ ID NO:1) of the FLNA sequence that correspond to amino acid residue positions 2561-2565 of the FLNA protein sequence as noted in the sequence provided at the web address: UniProtKB/Swiss-Prot entry P21333, FLNA-HUMAN, Filamin-A protein sequence. A FLNA-binding compound can inhibit the MOR-Gs coupling caused by agonist stimulation of the μ opioid receptor via interactions with filamin A, preferably in the 24^{th} repeat region. When co-administered with an opioid agonist, a FLNA-binding compound can enhance the analgesic effects and improve the treatment of pain.

As used herein, the term “candidate FLNA-binding compound” refers to a substance to be screened as a potential FLNA-binding compound. In preferred instances a FLNA-binding compound is also an opioid agonist. Additionally, a FLNA-binding compound can function in a combinatory manner similar to the combination of an opioid agonist and ultra-low-dose antagonist, wherein both FLNA and the mu-opioid receptor are targeted by a single entity.

As used herein, the term “opioid receptor” refers to a G protein coupled receptor, located in the central nervous system that interacts with opioids. More specifically, the p opioid receptor is activated by morphine causing analgesia, sedation, nausea, and many other side effects known to one of ordinary skill in the art.

As used herein, the term “opioid agonist” refers to a substance that upon binding to an opioid receptor can stimulate the receptor, induce G protein coupling and trigger a physiological response. More specifically, an opioid agonist is a morphine-like substance that interacts with MOR to produce analgesia.

As used herein, the term “opioid antagonist” refers to a substance that upon binding to an opioid receptor inhibits the function of an opioid agonist by interfering with the binding of the opioid agonist to the receptor.

As used herein an “analgesia effective amount” refers to an amount sufficient to provide analgesia or pain reduction to a recipient host.

As used herein the term “ultra-low-dose” or “ultra-low amount” refers to an amount of compound that when given in combination with an opioid agonist is sufficient to enhance the analgesic potency of the opioid agonist. More specifically, the ultra-low-dose of an opioid antagonist admixed with an opioid agonist in mammalian cells is an amount about 1000- to about 10,000,000-fold less, and preferably between about 10,000- and to about 1,000,000-fold less than the amount of opioid agonist.

As used herein an “FLNA-binding effective amount” refers to an amount sufficient to perform the functions described herein, such as inhibition of MOR-Gs coupling, prevention of the cAMP desensitization measure, inhibition of CREB S¹³³ phosphorylation and inhibition of any other cellular indices of opioid tolerance and dependence, which functions can also be ascribed to ultra-low-doses of certain opioid antagonists such as naloxone or naltrexone. When a polypeptide or FLNA-binding compound of the invention interacts with FLNA, an FLNA-binding effective amount can be an ultra-low amount or an amount higher than an ultra-low-dose as the polypeptide or FLNA-binding compound will not antagonize the opioid receptor and compete with the agonist, as occurs with known opioid antagonists such as naloxone or naltrexone in amounts greater than ultra-low-doses. More preferably, when a polypeptide or VAKGL-binding compound of the present invention both interacts with FLNA and is an agonist of the mu opioid receptor, an FLNA-binding effective amount is an amount higher than an ultra-low-dose and is a sufficient amount to activate the mu opioid receptor.

As used herein the phrase “determining inhibition of the interaction of a mu opioid receptor with a Gs protein” refers to monitoring the cellular index of opioid tolerance and dependence caused by chronic or high-dose administration of opioid agonists to mammalian cells. More specifically, the mu opioid receptor-Gs coupling response can be identified by measuring the presence of the Gas (stimulatory) subunit, the interaction of MOR with the G protein complexes and formation of Gs-MOR coupling, the interaction of the Gβγ protein with adenylyl cyclase types II and IV, loss of inhibition or outright enhancement of cAMP accumulation, and the activation of CREB via phosphorylation of S¹³³.

As used herein the term “naloxone/naltrexone positive control” refers to a positive control method comprising steps discussed in a method embodiment, wherein the candidate FLNA-binding compound is a known opioid antagonist administered in an ultra-low amount, preferably naloxone or naltrexone.

As used herein the term “FLNA-binding compound negative control” refers to a negative control method comprising steps discussed in a method embodiment, wherein the candidate FLNA-binding compound is absent and the method is carried out in the presence of only opioid agonist.

As used herein the term “pharmacophore” is not meant to imply any pharmacological activity. The term refers to chemical features and their distribution in three-dimensional space that constitutes and epitomizes the preferred requirements for molecular interaction with a receptor (U.S. Pat. No. 6,034,066).

### DETAILED DESCRIPTION OF THE INVENTION

It should be understood that the present disclosure is to be considered as an exemplification of the present invention, and is not intended to limit the invention to the specific embodiments illustrated. It should be further understood that the title of this section of this application (“Detailed Description of the Invention”) relates to a requirement of the United States Patent Office, and should not be found to limit the subject matter disclosed herein.

The present invention contemplates a compound that binds to FLNA and also stimulates the p opioid receptor (MOR), and method of its use to provide analgesia. A contemplated compound can inhibit MOR-Gs coupling through interactions with FLNA and/or the μ opioid receptor (MOR).

In another aspect of the present invention, a contemplated compound prevents the morphine-induced Gs protein coupling by MOR. That prevention of MOR-Gs coupling is believed to occur by preventing the interaction of filamin A and MOR. Downstream effects of preventing the MOR-Gs coupling include inhibition of cAMP accumulation and of cAMP Response Element Binding protein (CREB) activation in a manner resembling the activity of ultra-low-dose opioid antagonists naloxone and naltrexone.

In another aspect of the present invention, a FLNA-binding compound prevents the MOR-Gs coupling while itself activating MOR.

The data collected in organotypic striatal slice cultures demonstrate that after 7 days of twice daily 1-hour exposures to oxycodone, mu opioid receptors in striatum switch from Go to Gs coupling (compare vehicle to oxycodone conditions). In contrast, a compound contemplated herein did not cause a switch to Gs coupling despite its ability to stimulate mu opioid receptors as previously assessed by GTPγS binding that is blocked by beta-funaltrexamine, a specific mu opioid receptor antagonist. These data imply that these novel compounds provide the analgesic effects characteristic of opioid drugs but do not cause analgesic tolerance or dependence, and do not have the addictive potential of opioid drugs.

A compound contemplated by the present invention binds to an above-defined FLNA polypeptide as well as stimulates the μ A opioid receptor (MOR). A contemplated compound corresponds in structure to Formula I

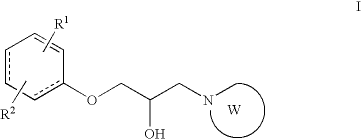

In a compound of Formula I, R¹ and R² are the same or different and are independently H, halogen, C₁-C₆ hydrocarbyl, C₁-C₆ acyl, C₁-C₆ hydrocarbyloxy and NR³R⁴ wherein R³ and R⁴ are the same or different and are H, C₁-C₄ hydrocarbyl, C₁-C₄ acyl, C₁-C₄ hydrocarbylsulfonyl, or R³ and R⁴ together with the depicted nitrogen form a 5-7-membered ring that optionally contains 1 or 2 additional hetero atoms that independently are nitrogen, oxygen or sulfur. W is a ring structure that contains 5 to 8 atoms in the ring including the depicted nitrogen. W and can optionally contain: a) 1 or 2 further hetero atoms that are independently oxygen, nitrogen or sulfur, and b) one or more substituent groups bonded to one or more ring atoms, in which the one or more substituents contain a total of up to 12 atoms selected from the group consisting of carbon, nitrogen, oxygen and sulfur, and mixtures thereof. A dotted line ( [symbol] ) represents an optional double bond. In regard to a contemplated compound, R¹ and R² are other than methyl and isopropyl, respectively, when W is N-morpholinyl or dimethyl-N-morpholinyl and the optional double bonds are absent.

In preferred practice, when one optional double bond is present, three double bonds are present so that the compound is a derivative of benzene. Thus, preferably, unless three double bonds are present none of the double bonds is present and the compound has a saturated ring.

In some preferred embodiments when three double bonds are present, one of R¹ and R² is H so that the compound is a disubstituted benzene derivative. In other embodiments, both of R¹ and R² are halogen, although not necessarily the same halogen. Thus, a compound containing a fluoro and a bromo group is contemplated, as are a compound containing a chloro and a bromo group, a compound containing two fluoro groups, two chloro groups, two bromo groups and two iodo groups.

Where one or both of R¹ and R² is NR³R⁴, it is preferred that R³ and R⁴ are the same C₁-C₄ hydrocarbyl, and more preferably both of R³ and R⁴ are methyl (C₁). It is also preferred that only on e of R¹ and R² be NR³R⁴, and that the other be H.

Where one or both of R¹ and R² is C₁-C₄ acyl, it is preferred that only one of R¹ and R² is C₁-C₄ acyl and the other is H. A preferred C₁-C₄ acyl group is an acetyl group [CH₃C(O)—].

In another preferred embodiment, a contemplated compound corresponds in structure to Formula Ia

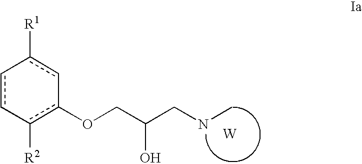

wherein

R¹ and R² are the same or different and are independently H, or C₁-C₆ hydrocarbyl;

W is a ring structure that contains 5 to 8 atoms in the ring including the depicted nitrogen, and can optionally contain
a) 1 or 2 further hetero atoms that are independently oxygen, nitrogen or sulfur, and
b) one or more substituent groups bonded to one or more ring atoms, in which the one or more substituent contain a total of up to 12 atoms selected from the group consisting of carbon, nitrogen, oxygen and sulfur, and mixtures thereof;

a dotted line ( [symbol] ) represents 1, 2, or 3 optional double bonds,

with the proviso that R¹ and R² are other than methyl and isopropyl, respectively, when W is N-morpholinyl

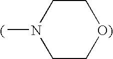

or dimethyl-N-morpholinyl

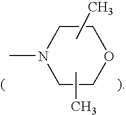

In preferred practice here, W further includes one or more substituent groups bonded to one or more ring atoms, in which those one or more substituents contain a total of up to 12 atoms selected from the group consisting of carbon, nitrogen, oxygen and sulfur, and mixtures thereof.

In one preferred embodiment, a compound of Formula I has the structure of Formula II, whereas in another preferred embodiment, a compound of Formula I has the structure of a compound of Formula III. R¹ and R² and W are as previously defined in a compound of both Formulas II and III.

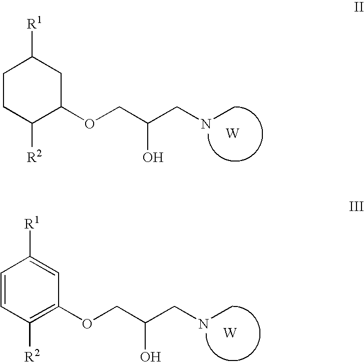

More preferably, the R¹ and R² of a compound of Formula II contain 3 to 5 carbon atoms, whereas for a compound of Formula III, R¹ is H and R² contains 3 to 5 carbon atoms.

In a compound of either formula II or III, W can optionally contain 1 or 2 further hetero atoms that are independently oxygen, nitrogen or sulfur, and more preferably still contains at least one such hetero atom. It is also preferred that W further includes one or more substituent groups bonded to one or more ring atoms, in which the one or more substituents contain a total of up to 12 atoms selected from the group consisting of carbon, nitrogen, oxygen and sulfur, and mixtures thereof. Illustrative W substituents are illustrated below, wherein the wavy line indicates the position of the bond between W and the remainder of the compound.

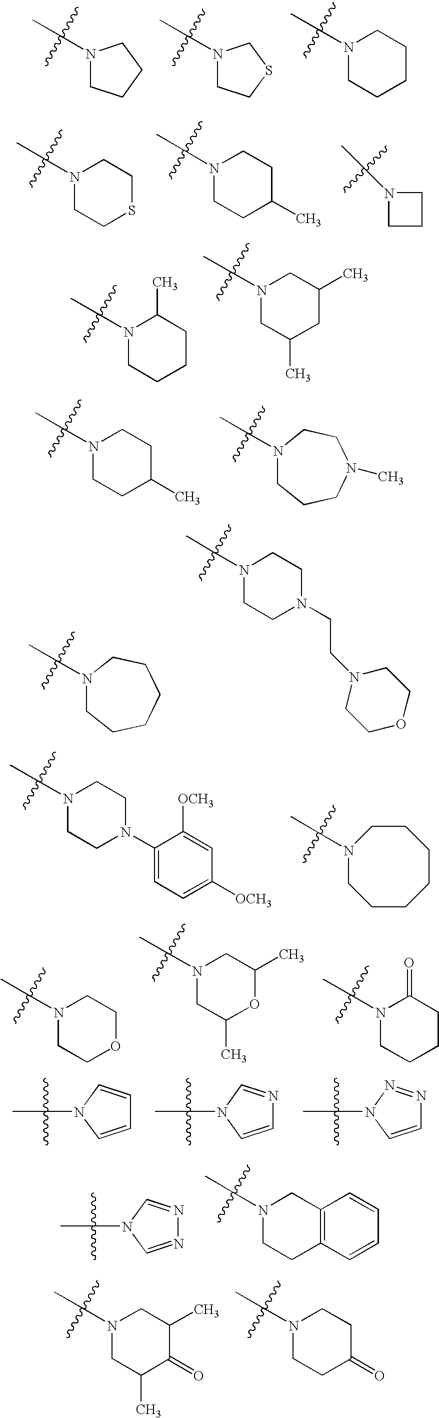

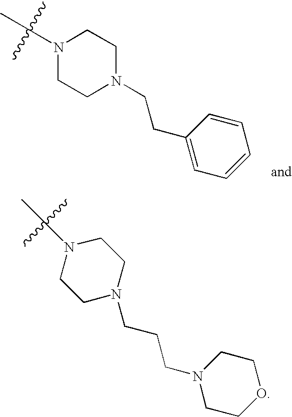

Use of a compound of Formula II selected from the group consisting of one or a mixture of the following in a contemplated method of reducing pain is particularly preferred

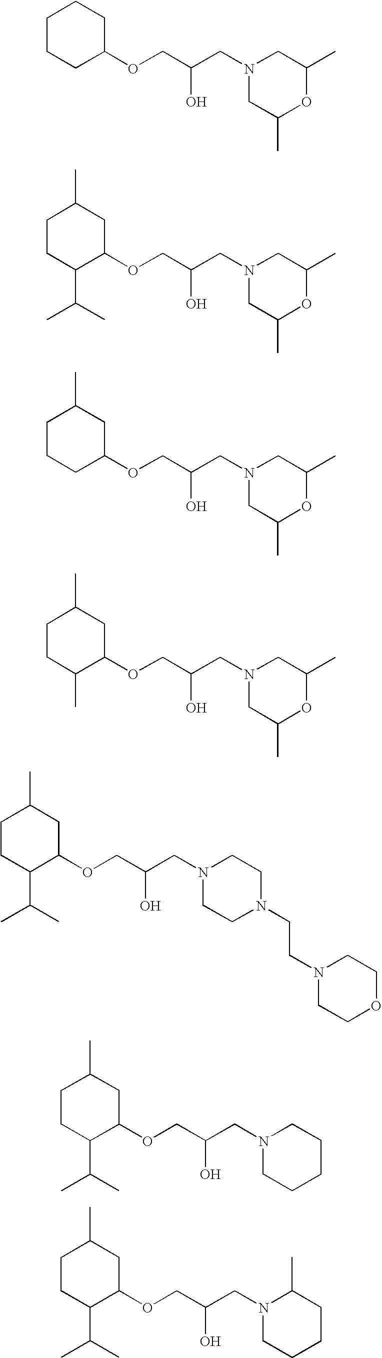

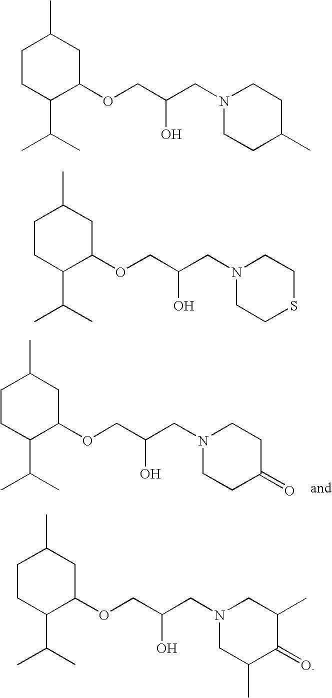

Of the above compounds, the following are themselves particularly preferred:

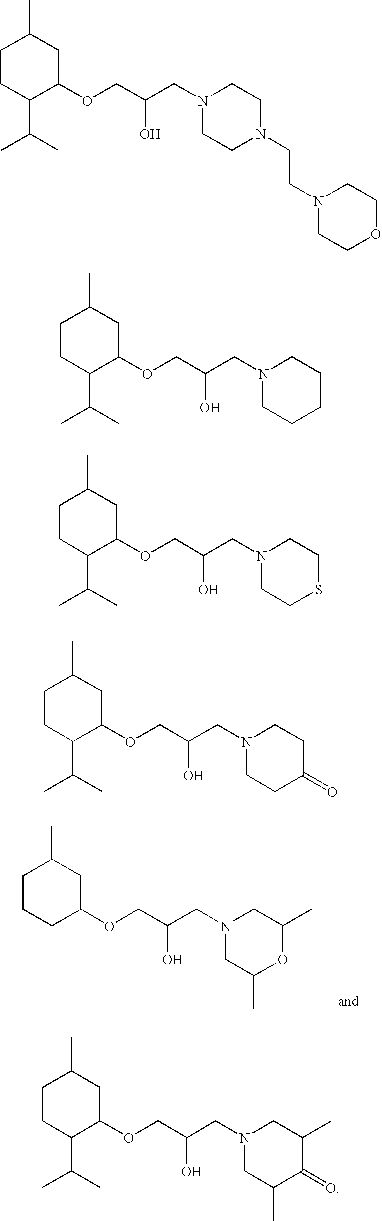

Of the above compounds, the following compound that is referred to in the assays described hereinafter as compound A0011 is more particularly preferred.

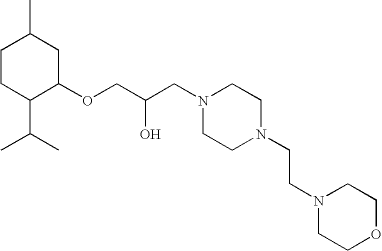

A particularly preferred compound of Formula III is selected from the group consisting of

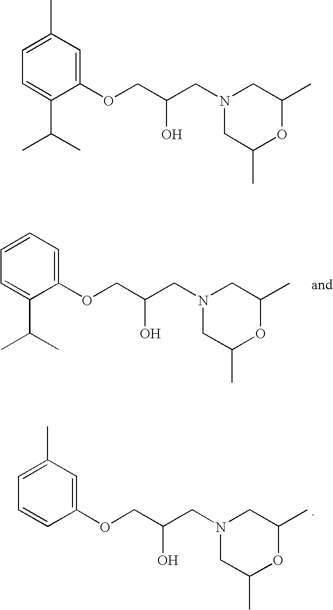

In another aspect, a contemplated compound is selected in part using a method for determining the ability of a candidate FLNA-binding compound, other than naloxone or naltrexone, to inhibit the interaction of the mu opioid receptor with filamin A (FLNA) and thereby prevent the mu opioid receptor from coupling to Gs proteins (Gs). That method comprises the steps of: (a) admixing the candidate FLNA-binding compound (alone if such FLNA-binding compound also stimulates MOR or with a MOR agonist otherwise) with mammalian cells that contain the mu opioid receptor and FLNA in their native conformations and relative orientations, the opioid agonist being present in an agonist effective amount and/or being administered in a repeated, chronic manner the FLNA-binding compound being present in an FLNA-binding effective amount; and (b) determining inhibition of the interaction of the mu opioid receptor with the G protein by analysis of the presence or the absence of the Gas subunit of Gs protein, wherein the absence of the Gas subunit indicates inhibition of the interaction of the mu opioid receptor with the Gs protein.

In one aspect, the analysis of Gs protein coupling by the mu opioid receptor and downstream effects elicited by admixing mammalian cells with a before-defined compound can be conducted by any one or more of several methods such as for example co-immunoprecipitation of Ga proteins with MOR, Western blot detection of MOR in immunoprecipitates, and densitometric quantification of Western blots.

### Pharmaceutical Composition

A pharmaceutical composition is contemplated that contains an analgesia effective amount of a compound of Formula I, Formula Ia, Formula II, or Formula III dissolved or dispersed in a physiologically tolerable carrier. Such a composition can be administered to mammalian cells in vitro as in a cell culture, or in vivo as in a living, host mammal in need.

A contemplated composition is typically administered a plurality of times over a period of days. More usually, a contemplated composition is administered a plurality of times in one day.

As is seen from the data that follow, a contemplated compound is active in the assays studies at micromolar amounts. In the laboratory mouse tail flick test, contemplated compound A0011 exhibited peak activity at about ten minutes using a dose of 56 mg/kg. Morphine administered at the same dose exhibited a slightly greater antinoniceptive effect at twenty minutes. It is thus seen that the contemplated compounds are quite active and potent, and that a skilled worker can readily determine an appropriate dosage level, particularly in view of the relative activity of a contemplated compound compared to orally administered morphine.

A contemplated composition described herein can be used in the manufacture of a medicament that is useful at least for lessening or reducing pain in a mammal that is in need.

A contemplated pharmaceutical composition can be administered orally (perorally), parenterally, by inhalation spray in a formulation containing conventional nontoxic pharmaceutically acceptable carriers, adjuvants, and vehicles as desired. The term parenteral as used herein includes subcutaneous injections, intravenous, intramuscular, intrasternal injection, or infusion techniques. Formulation of drugs is discussed in, for example, Hoover, John E., Remington's Pharmaceutical Sciences, Mack Publishing Co., Easton, Pa.; 1975 and Liberman, H. A. and Lachman, L., Eds., Pharmaceutical Dosage Forms, Marcel Decker, New York, N.Y., 1980.

Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution, phosphate-buffered saline. Liquid pharmaceutical compositions include, for example, solutions suitable for parenteral administration. Sterile water solutions of an active component or sterile solution of the active component in solvents comprising water, ethanol, or propylene glycol are examples of liquid compositions suitable for parenteral administration.

In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

Sterile solutions can be prepared by dissolving the active component in the desired solvent system, and then passing the resulting solution through a membrane filter to sterilize it or, alternatively, by dissolving the sterile compound in a previously sterilized solvent under sterile conditions.

Solid dosage forms for oral administration can include capsules, tablets, pills, powders, and granules. In such solid dosage forms, the compounds of this invention are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets and pills can additionally be prepared with enteric coatings.

A mammal in need of treatment and to which a pharmaceutical composition containing a contemplated compound is administered can be a primate such as a human, an ape such as a chimpanzee or gorilla, a monkey such as a cynomolgus monkey or a macaque, a laboratory animal such as a rat, mouse or rabbit, a companion animal such as a dog, cat, horse, or a food animal such as a cow or steer, sheep, lamb, pig, goat, llama or the like.

Where in vitro mammalian cell contact is contemplated, a CNS tissue culture of cells from an illustrative mammal is often utilized, as is illustrated hereinafter. In addition, a non-CNS tissue preparation that contains opioid receptors such as guinea pig ileumcan also be used.

Preferably, the pharmaceutical composition is in unit dosage form. In such form, the composition is divided into unit doses containing appropriate quantities of the active urea. The unit dosage form can be a packaged preparation, the package containing discrete quantities of the preparation, for example, in vials or ampules.

### EXAMPLES

The present invention is described in the following examples which are set forth to aid in the understanding of the invention, and should not be construed to limit in any way the invention as defined in the claims which follow thereafter.

The experiments described herein were carried out on organotypic striatal slices from male Sprague Dawley rats (200 to 250 g) purchased from Taconic (Germantown, N.Y.). Rats were housed two per cage and maintained on a regular 12-hour light/dark cycle in a climate-controlled room with food and water available ad libitum and sacrificed by rapid decapitation. All data are presented as mean±standard error of the mean. Treatment effects were evaluated by two-way ANOVA followed by Newman-Keul's test for multiple comparisons. Two-tailed Student's t test was used for post hoc pairwise comparisons. The threshold for significance was p<0.05.

The following Table of Correspondence shows the structures of the compounds discussed herein and their identifying numbers.

Table of Correspondence

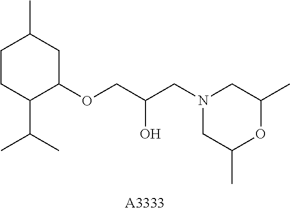

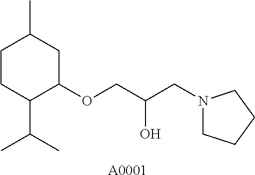

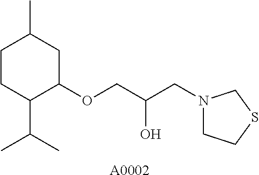

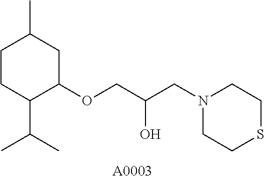

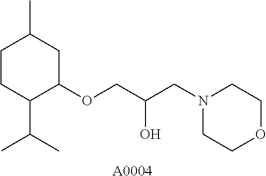

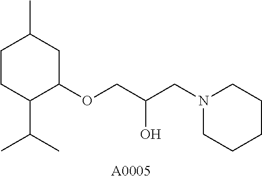

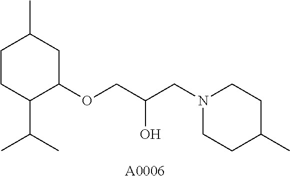

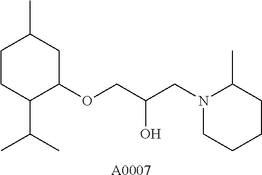

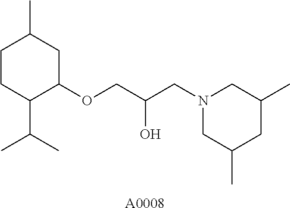

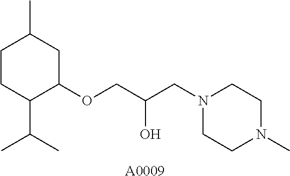

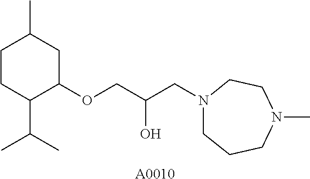

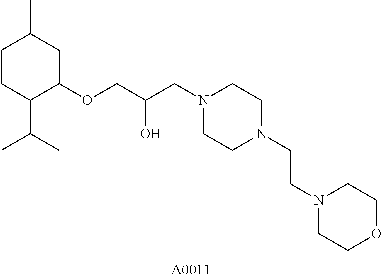

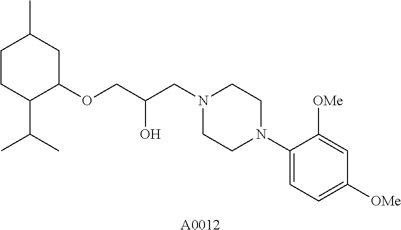

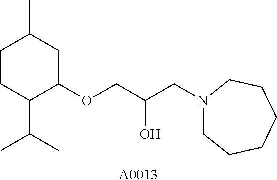

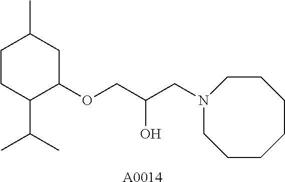

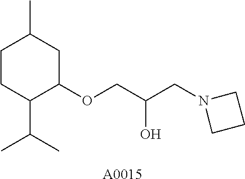

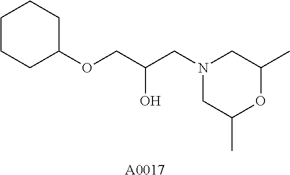

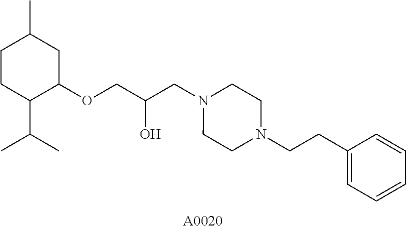

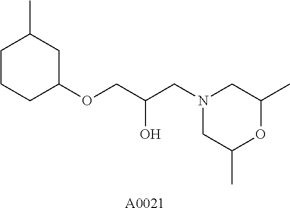

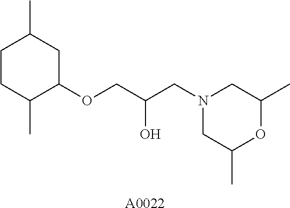

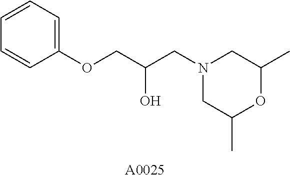

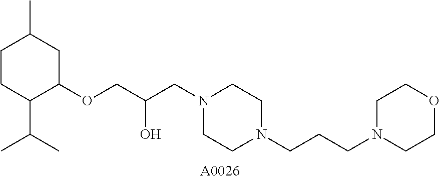

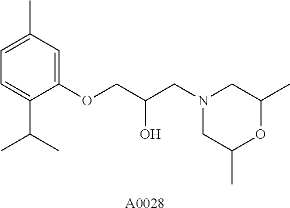

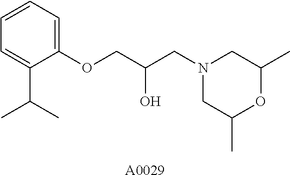

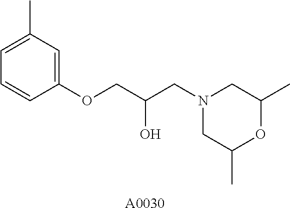

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

### Example 1

### MOR Agonist Activity Using GTPγS Binding Assay

To assess the mu opiate receptor (MOR) agonist activity of positive compounds from the FLNA screening, compounds were tested in a [³⁵S]GTPγS binding assay using striatal membranes. Our previous study has shown that in striatal membranes, activation of MOR leads to an increase in [³⁵S]GTPγS binding to Gαo (Wang et al., 2005 Neuroscience 135:247-261).

Striatal tissue was homogenized in 10 volumes of ice cold 25 mM HEPES buffer, pH 7.4, which contained 1 mM EGTA, 100 mM sucrose, 50 μg/ml leupeptin, 0.04 mM PMSF, 2 μg/ml soybean trypsin inhibitor and 0.2% 2-mercaptoethanol. The homogenates were centrifuged at 800×g for 5 minutes and the supernatants were centrifuged at 49,000×g for 20 minutes. The resulting pellets were suspended in 10 volume of reaction buffer, which contained 25 mM HEPES, pH 7.5, 100 mM NaCl, 50 μg/ml leupeptin, 2 μg/ml soybean trypsin inhibitor, 0.04 mM PMSF and 0.02% 2-mercaptomethanol.

The resultant striatal membrane preparation (200 μg) was admixed and maintained (incubated) at 30° C. for 5 minutes in reaction buffer as above that additionally contained 1 mM MgCl₂ and 0.5 nM [⁵S]GTPγS (0.1 μCi/assay, PerkinElmer Life and Analytical Sciences) in a total volume of 2501 and continued for 5 minutes in the absence or presence of 0.1-10 μM of an assayed compound of interest. The reaction was terminated by dilution with 750 μl of ice-cold reaction buffer that contained 20 mM MgCl₂ and 1 mM EGTA and immediate centrifugation at 16,000×g for 5 minutes.

The resulting pellet was solubilized by sonicating for 10 seconds in 0.5 ml of immunoprecipitation buffer containing 0.5% digitonin, 0.2% sodium cholate and 0.5% NP-40. Normal rabbit serum (1 μl) was added to 1 ml of lysate and incubated at 25° C. for 30 minutes. Nonspecific immune complexes were removed by incubation with 25 μl of protein A/G-conjugated agarose beads at 25° C. for 30 minutes followed by centrifugation at 5,000×g at 4° C. for 5 minutes. The supernatant was divided and separately incubated at 25° C. for 30 minutes with antibodies raised against Gαo proteins (1:1,000 dilutions).

The immunocomplexes so formed were collected by incubation at 25° C. for 30 minutes with 40 μl of agarose-conjugated protein A/G beads and centrifugation at 5,000×g at 4° C. for 5 minutes. The pellet was washed and suspended in buffer containing 50 mM Tris-HCl, pH 8.0, and 1% NP-40. The radioactivity in the suspension was determined by liquid scintillation spectrometry. The specificity of MOR activation of [³⁵S]GTPγS binding to Gαo induced by a selective compound was defined by inclusion of 1 μM β-funaltrexamine (β-FNA; an alkylating derivative of naltrexone that is a selective MOR antagonist). DAMGO (H-Tyr-D-Ala-Gly-N-MePhe-Gly-OH; 1 or 10 μM) was used as a positive control.

The results of this study are shown in the Table below.

FLNA-Binding Compound MOR Agonist Activity

| FLNA- | Concentration of FLNA-Binding Compound as Agonist | | | | | |
|---|---|---|---|---|---|---|
| Binding | | | 1 μM + | % DAMGO | % DAMGO | % DAMGO + |
| Compound | 0.1 μM | 1 μM | BFNA | (0.1 μM) | (1 μM) | BFNA |
| A3333 | 170.7% | 328.3% | 65.9% | 88.9% | 101.0% | 136.7% |
| A0001 | 94.3% | 181.7% | 22.2% | 63.1% | 78.9% | 83.8% |
| A0002 | 155.6% | 199.4% | 6.5% | 104.1% | 86.6% | 24.5% |
| A0003 | 176.8% | 276.0% | 17.1% | 118.3% | 119.9% | 64.5% |
| A0004 | 97.4% | 144.2% | 86.0% | 55.2% | 55.6% | 130.9% |
| A0005 | 179.7% | 239.2% | 23.5% | 105.0% | 89.6% | 45.1% |
| A0006 | 170.0% | 190.9% | 18.2% | 113.8% | 82.9% | 68.7% |
| A0007 | 102.0% | 221.9% | 40.4% | 68.3% | 96.4% | 152.5% |
| A0008 | 163.8% | 235.0% | 133.9% | 109.6% | 102.1% | 505.3% |
| A0009 | 70.2% | 126.4% | 93.9% | 39.8% | 48.7% | 142.9% |
| A0010 | 277.2% | 319.0% | 190.3% | 161.9% | 119.5% | 365.3% |
| A0011 | 236.3% | 287.5% | 47.0% | 158.2% | 124.9% | 177.4% |
| A0012 | 149.3% | 185.7% | 122.4% | 99.9% | 80.7% | 461.9% |
| A0013 | 102.1% | 164.8% | 86.1% | 57.8% | 63.6% | 131.1% |
| A0014 | 147.0% | 174.9% | 140.8% | 83.2% | 67.5% | 214.3% |
| A0015 | 110.9% | 150.1% | 62.5% | 64.8% | 56.2% | 120.0% |
| A0017 | 161.9% | 246.0% | 65.2% | 96.9% | 100.4% | 187.9% |
| A0020 | 168.6% | 217.4% | 67.4% | 100.9% | 88.7% | 194.2% |
| A0021 | 133.3% | 275.3% | 12.1% | 79.8% | 112.4% | 34.9% |
| A0022 | 154.1% | 216.0% | 28.0% | 90.0% | 80.9% | 53.7% |
| A0025 | 58.6% | 138.7% | 52.2% | 33.2% | 54.5% | 198.5% |
| A0026 | 140.7% | 179.8% | 120.8% | 79.7% | 70.7% | 459.3% |
| A0028 | 143.6% | 187.7% | 116.7% | 81.3% | 73.8% | 443.7% |
| A0029 | 173.8% | 206.5% | 22.3% | 98.4% | 81.2% | 84.8% |
| A0030 | 133.4% | 287.8% | 165.2% | 75.5% | 113.2% | 628.1% |

### Example 2

### FITC-NLX-Based FLNA Screening Assay

A. Streptavidin-Coated 96-Well Plates

Streptavidin-coated 96-well plates (Reacti-Bind™ NeutrAvidin™ High binding capacity coated 96-well plate, Pierce-ENDOGEN) are washed three times with 200 μl of 50 mM Tris HCl, pH 7.4 according to the manufacturer's recommendation.

B. N-Biotinylated VAKGL Pentapeptide (Bn-VAKGL) (SEQ ID NO: 1)

En-VAKGL peptide (0.5 mg/plate) is dissolved in 50 μl DMSO and then added to 4450 μl of 50 mM Tris HCl, pH 7.4, containing 100 mM NaCl and protease inhibitors (binding medium) as well as 500 μl superblock in PBS (Pierce-ENDOGEN) [final concentration for DMSO: 1%].

C. Coupling of Bn-VAKGL Peptides to Streptavidin-Coated Plate

The washed streptavidin-coated plates are contacted with 5 μg/well of Bn-VAKGL (100 μl) for 1 hour (incubated) with constant shaking at 25° C. [50 of Bn-VAKGL peptide solution from B+50 μl binding medium, final concentration for DMSO: 0.5%]. At the end of the incubation, the plate is washed three times with 200 μl of ice-cold 50 mM Tris HCl, pH 7.4.

D. Binding of FITC-Tagged Naloxone [FITC-NLX] to VAKGL

Bn-VAKGL coated streptavidin plates are incubated with 10 nM fluorescein isothiocyanate-labeled naloxone (FITC-NLX; Invitrogen) in binding medium (50 mM Tris HCl, pH 7.4 containing 100 mM NaCl and protease inhibitors) for 30 minutes at 30° C. with constant shaking. The final assay volume is 100 μla. At the end of incubation, the plate is washed twice with 100 μl of ice-cold 50 mM Tris, pH 7.4. The signal, bound-FITC-NLX is detected using a DTX-880 multi-mode plate reader (Beckman).

E. Screening of Medicinal Chemistry Analogs

The compounds are first individually dissolved in 25% DMSO containing 50 mM Tris HCl, pH 7.4, to a final concentration of 1 mM (assisted by sonication when necessary) and then plated into 96-well compound plates. To screen the medicinal chemistry analogs (new compounds), each compound solution (1 μl) is added to the Bn-VAKGL coated streptavidin plate with 50 μl/well of binding medium followed immediately with addition of 50 μl of FITC-NLX (total assay volume/well is 100 μl). The final screening concentration for each compound is 10 μM.

Each screening plate includes vehicle control (total binding) as well as naloxone (NLX) and/or naltrexone (NTX) as positive controls. Compounds are tested in triplicate or quadruplicate. Percent inhibition of FITC-NLX binding for each compound is calculated [(Total FITC-NLX bound in vehicle−FITC-NLX bound in compound)/Total FITC-NLX bound in vehicle]×100%]. To assess the efficacies and potencies of the selected compounds, compounds that achieve approximately 60-701 inhibition at 10 μM are screened further at 1 and 0.1 μM concentrations.

The results of this screening assay are shown in the table below.

FLNA Peptide Binding Assay

| | |
|---|---|
| FLNA-binding | Concentration of FLNA-binding Compound |

| Compound | 0.01 μM | 0.1 μM | 1 μM |
|---|---|---|---|
| 3333 | 40.4% | 48.5% | 54.2% |
| A0001 | 39.7% | 45.6% | 52.4% |
| A0002 | 38.7% | 43.7% | 49.9% |
| A0003 | 21.3% | 31.6% | 37.4% |
| A0004 | 40.0% | 43.7% | 47.6% |
| A0005 | 34.2% | 38.2% | 43.8% |
| A0006 | 37.9% | 43.5% | 47.5% |
| A0007 | 39.2% | 46.2% | 52.9% |
| A0008 | 34.5% | 33.5% | 39.8% |
| A0009 | 26.4% | 37.8% | 38.9% |
| A0010 | 36.0% | 36.5% | 39.0% |
| A0011 | 45.7% | 51.1% | 52.8% |
| A0012 | 39.7% | 49.6% | 54.4% |
| A0013 | 30.2% | 40.2% | 47.7% |
| A0014 | 33.8% | 39.7% | 44.7% |
| A0015 | 36.3% | 46.8% | 55.0% |
| A0017 | 29.8% | 38.6% | 44.0% |
| A0020 | 37.8% | 38.8% | 45.8% |
| A0021 | 36.8% | 43.4% | 49.5% |
| A0022 | 41.9% | 49.7% | 56.8% |
| A0025 | 39.0% | 49.8% | 53.2% |
| A0026 | 36.4% | 42.4% | 49.2% |
| A0028 | 39.5% | 43.8% | 50.5% |
| A0029 | 44.4% | 44.4% | 50.8% |
| A0030 | 35.6% | 44.4% | 48.9% |

### Example 3

### Tail-Flick Test

The mouse “tail flick” test was used to assay the relative antinociceptive activity of compositions containing a compound to be assayed. This assay was substantially that disclosed by Xie et al., 2005 J. Neurosci 25:409-416.

The mouse hot-water tail-flick test was performed by placing the distal third of the tail in a water bath maintained at 52° C. The latency until tail withdrawal from the bath was determined and compared among the treatments. A 10 second cutoff was used to avoid tissue damage. Data are converted to percentage of antinociception by the following formula: (response latency−baseline latency)/(cutoff−baseline latency)×100 to generate dose-response curves. Linear regression analysis of the log dose-response curves was used to calculate the A₅₀ (dose that resulted in a 50% antinociceptive effect) doses and the 95% confidence intervals (CIs). Relative potency was determined as a ratio of the A₅₀ values. The significance of the relative potency and the confidence intervals are determined by applying the t test at p<0.05.

To assess tolerance to the antinociceptive effect, the compound was administered twice daily for 7 days at an A₉₀ dose (dose that results in a 90% antinociceptive effect in the 52° C. warm-water tail-flick test), and the tail-flick test was performed daily after the a.m. dose. A significant reduction in tail-flick latency on subsequent days compared to the Day 1 administration of the A₉₀ dose indicates antinociceptive tolerance.

Orally administered morphine exhibited an A₅₀ value of 61.8 (52.4−72.9) mg/kg, and a mean maximum antinoniception amount of about 43% at 56 mg/kg at about 20 minutes. Orally administered compound A0011 exhibited a mean maximum antinoniception amount of about 38% at 56 mg/kg at about 10 minutes.

### Example 4

### Dependence Test

On day 8, 16-20 hours after the last administration of an assay composition, animals were given naloxone to precipitate withdrawal (10 mg/kg, s.c.) before being placed in an observation chamber for 1 hour. A scale adapted from MacRae et al., 1997 Psychobiology 25:77-82 was used to quantify four categories of withdrawal behaviors: “wet dog” shakes, paw tremors, mouth movements, and ear wipes. Scores are summed to yield a total withdrawal score across the 1-hour test.

### Example 5

### Relative Gs/Go Switching

In this set of studies, the rat brain slice organotypic culture methods were modified from those published previously (Adamchik et al., 2000 Brain Res Protoc 5:153-158; Stoppini et al., 1991 J Neurosci Methods 37:173-182). Striatal slices (200 μM thickness) were prepared using a McIlwain tissue chopper (Mickle Laboratory Engineering Co., Surrey, UK). Slices were carefully transferred to sterile, porous culture inserts (0.4 μm, Millicell-CM) using the rear end of a glass Pasteur pipette. Each culture insert unit contained 2 slices and was placed into one well of the 12-well culture tray. Each well contain 1.5 ml of culture medium composed of 50% MEM with Earl's salts, 2 mM L-glutamine, 25% Earl's balanced salt solution, 6.5 g/l D-glucose, 20% fetal bovine serum, 5% horse serum, 25 mM HEPES buffer, 50 mg/ml streptomycin and 50 mg/ml penicillin. The pH value was adjusted to 7.2 with HEPES buffer.

Cultures were first incubated for 2 days to minimize the impact of injury from slice preparation. Incubator settings throughout the experiment were 36° C. with 5% CO₂. To induce tolerance, culture medium was removed and the culture insert containing the slices was gently rinsed twice with warm (37° C.) phosphate-buffered saline (pH 7.2) before incubation in 0.1% fetal bovine serum-containing culture medium with 100 μM morphine for 1 hour twice daily (at 9-10 AM and 3-4 PM) for 7 days.

Slices were returned to culture medium with normal serum after each drug exposure. Tissues were harvested 16 hours after the last drug exposure by centrifugation.

For determination of MOR-G protein coupling, slices were homogenized to generate synaptic membranes. Synaptic membranes (400 μg) were incubated with either 10 μM oxycodone or Kreb's-Ringer solution for 10 minutes before solubilization in 250 μl of immunoprecipitation buffer (25 mM HEPES, pH 7.5; 200 mM NaCl, 1 mM EDTA, 50 μg/ml leupeptin, 10 μg/ml aprotinin, 2 μg/ml soybean trypsin inhibitor, 0.04 mM PMSF and mixture of protein phosphatase inhibitors). Following centrifugation, striatal membrane lysates were immunoprecipitated with immobilized anti-Gαs/olf or -Gαo conjugated with immobilized protein G-agarose beads. The level of MOR in anti-Gαs/olf or -Gαo immunoprecipitates was determined by Western blotting using specific anti-MOR antibodies.

To measure the magnitude of MOR-mediated inhibition of cAMP production, brain slices were incubated with Kreb's-Ringer (basal), 1 μM DAMGO, 1 μM forskolin or 1 μM DAMGO+1 μM forskolin for 10 minutes at 37° C. in the presence of 100 μM of the phosphodiesterase inhibitor IBMX. Tissues were homogenized by sonication and protein precipitated with 1M TCA. The supernatant obtained after centrifugation was neutralized using 50 mM Tris, pH 9.0. The level of cAMP in the brain lysate was measured by a cAMP assay kit (PerkinElmer Life Science, Boston) according to manufacturer's instructions.

| | | | Gs/Go-Coupled |
|---|---|---|---|
| Condition | Gs/olf | Go | Ratio |
| Vehicle | | | |
| Average | 330.7 | 1996.4 | 0.173 |
| SEM | 34.6 | 192.0 | 0.34 |
| Oxycodone, | | | |
| 10 μM | | | |
| Average | 1425.2 | 900.4 | 1.588 |
| SEM | 77.8 | 26.2 | 0.103 |
| 3333, | | | |
| 10 μM | | | |
| Average | 1079.0 | 1426.3 | 0.761 |
| SEM | 66.2 | 46.5 | 0.070 |
| 3333, | | | |
| 100 μM | | | |
| Average | 1064.8 | 1418.8 | 0.756 |
| SEM | 94.2 | 82.9 | 0.077 |

### Compound Synthesis

A contemplated compound can be readily synthesized. An illustrative synthetic scheme is shown below and more detailed syntheses are set out hereinafter.

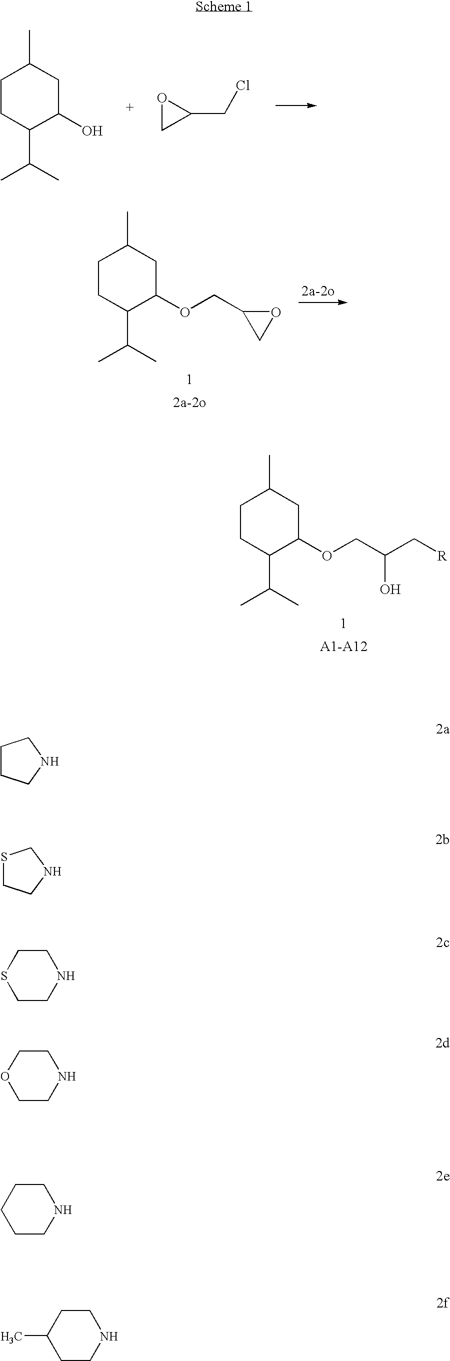

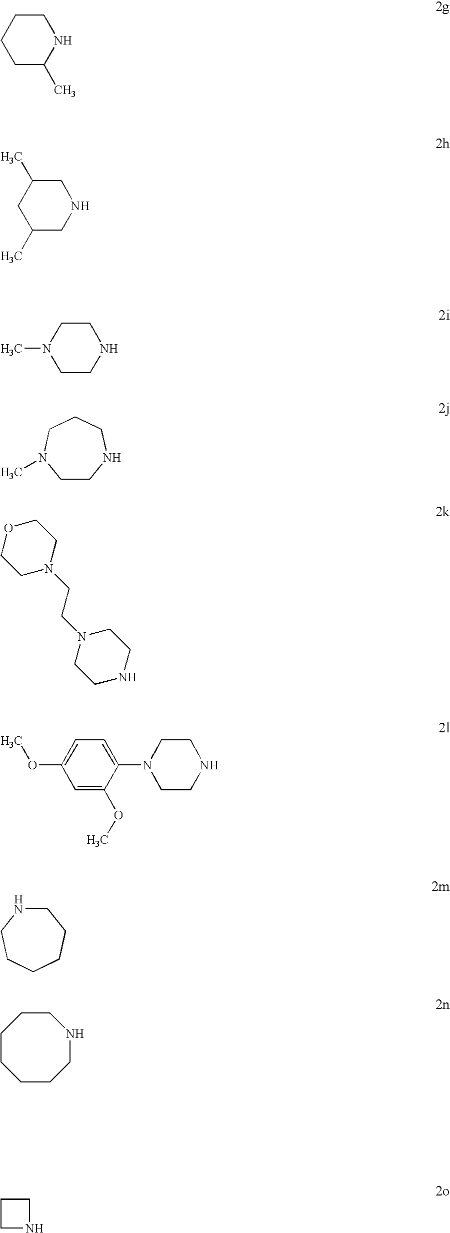

### Preparation of Compound 1

A flask was charged with D-menthol (10 g, 64 mmol), 40 mL toluene, and AlCl3 (0.68 g, 5.12 mmol). The temperature of the mixture was raised to 160° C., then epichlorohydrin (5.9 g, 64 mmol) was added with stirring for 1 hour. Next, NaOH (50%) (10.24 g, 128 mmol) was added with stirring at a temperature of 75° C. overnight (about 18 hours). Following this treatment, 5 mL of water was added into the mixture, which was then extracted three times with ethyl acetate (15 mL). The organic phase washings were combined, then the combined organic phase was dried and concentrated to obtain crude product. The crude product was purified by silica gel column to obtain the purified product, which was a colorless oil (TLC confirmed, 12.8 g, yield: 94%).

### Preparation of Compound 3333

A mixture of compound I (490 mg, 2.311 mmol), 2,6-dimethylmorpholine (532 mg, 4.632 mmol) and H₂O (0.4 ml) was stirred overnight (about 18 hours) at room temperature. The resulting mixture was extracted with ethyl acetate, washed with brine, dried with anhydrous sodium sulfate, and concentrated under vacuum to afford 701 mg of crude product as a yellow liquid. The crude product was purified via column chromatography to afford 290 mg of the desired product (yield: 38.4%) and 111.9 mg of an unidentified isomer (yield: 14.7%).

### Preparation of Compound A0015

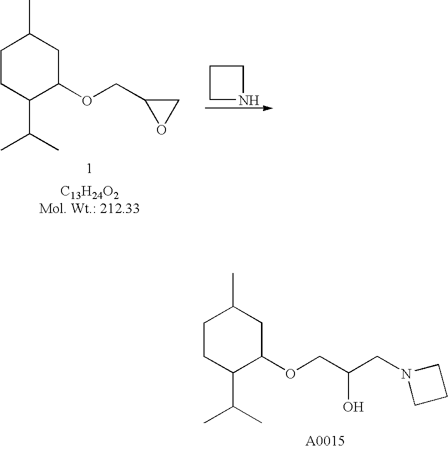

A mixture of compound I (123 mg, 0.58 mmol) and azetidine (33 mg, 0.58 mmol) in 0.1 mL of water was stirred at room temperature overnight (about 18 hours). Next, 3 mL of H₂O and 5 mL EA was added into the mixture. Next, the organic phase was separated, dried and concentrated to obtain an oily residue. Then, the crude product was purified by silica gel column (DCM:CH₃OH=20:1) to give 72 mg of purified product (LC-MS confirmed, yield 46%).

### Preparation of Compound A0017

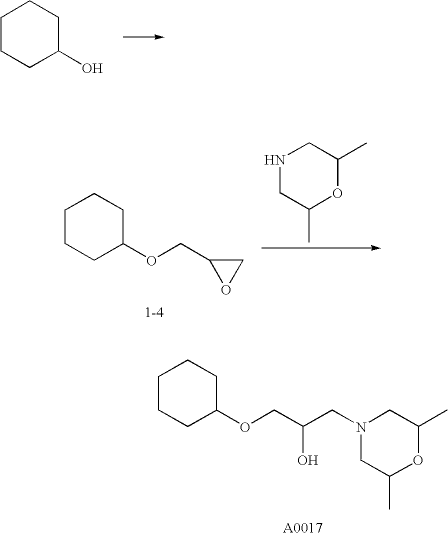

### Preparation of Compound 1-4

Under nitrogen atmosphere, into a reaction flask were added cyclohexanol (1 g, 10 mmol) and toluene (4 ml) at room temperature. Then, anhydrous aluminum chloride (0.1 g, 0.8 mmol) was added and dissolved under stirring and the temperature was raised to 116° C. Into the solution was added dropwise a solution of epichlorohydrin (1.37 g, 10 mmol) in toluene (2 ml). After the addition, the mixture was stirred at the same temperature for 1 hour. Thereafter, the reaction mixture was cooled to 50° C. Under nitrogen atmosphere, into the reaction mixture was added a 50% aqueous sodium hydroxide solution (1.6 g) and stirred at 75° C. overnight (about 18 hours). The mixture was washed with water then the solvent was removed by evaporation to obtain the crude product. The crude product was purified by silica gel (eluted by PE) to afford 987 mg of purified product (1H NMR confirmed, yield 63.2%).

### Preparation of Compound A0035

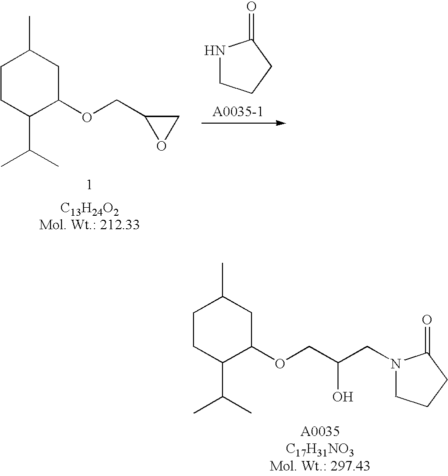

To a solution of compound A0035-1 (72 mg, 0.85 mmol) in 3 ml of dioxane was added 60% NaH (26 mg, 1.08 mmol), with stirring for 30 minutes. Next, compound I (100 mg, 0.47 mmol) in 2 ml of dioxane was added to the mixture with stirring at 100° C. overnight. Next, 8 ml of EA was added into the mixture, washed three times with H₂O (5 ml), then the organic phase was concentrated to get crude product (168 mg). The crude product was then purified to a colorless oil (38 mg, NMR and LCMS confirmed, yield: 1.3%, purity 96.6% by ELSD).

### Preparation of Compound A0036

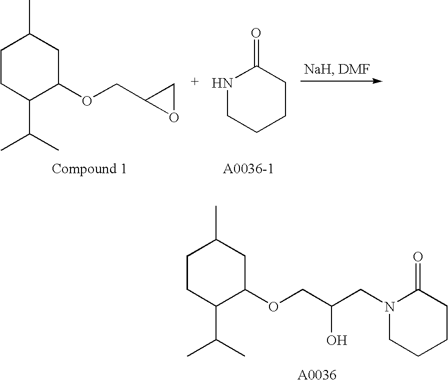

About 42 mg of Nail as a 60% dispersion in mineral oil was suspended in 2 ml absolute DMSO, stirred at room temperature for 10 minutes, admixed with A0036-1 (51.4 mg, 0.519 mmol) and stirred for 1 hour. Next, a solution of compound 1 (100 mg, 0.472 mmol) in 2 ml absolute DMSO was added drop-wise. The mixture was then stirred overnight (about 18 hours). TLC showed that some of the starting material remained. Next, the reaction mixture was heated to 55° C. and stirred for 4 hours.

Next, the reaction mixture was dissolved in ethyl acetate (EA), washed with water and brine, dried with anhydrous sodium sulfate and concentrated under vacuum to afford 90 mg of crude product.

The crude product was then purified via column chromatography two times (PE/EA=20/1 to EA) to afford 30 mg of the purified product (yield: 20%). The structure was confirmed by H NMR & MS, purity 93.6% by HPLC.

### Preparation of Compound A0038

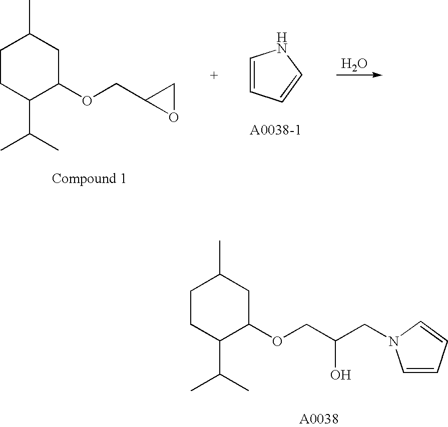

About 13 mg of NaH as 60% dispersion in mineral oil was suspended in 3 ml absolute DMF, stirred at room temperature for 10 minutes, then admixed with A0038-1 (34.8, 0.519 mmol) for 1 hour. Next, a solution of compound I (100 mg, 0.472 mmol) in 2 ml absolute DMF was added drop-wise with stirring overnight (about 18 hours). TLC showed the starting material remained. Next, the reaction mixture was heated to 45° C. and stirred for three more hours. TLC showed the starting material still remained. Next, 20 ml water was added to the reaction mixture followed by extraction with diethyl ether. The organic layers were saved and combined, then washed with 1 N HCl, then brine, then dried with anhydrous Na₂SO₄. The crude product was concentrated under vacuum to afford 108 mg of crude product and was purified via column chromatography (PE/EA=50/1 to PE/EA=20/1) to afford 16 mg of purified product (yield: 13.7%).

### Preparation of Compound A0031

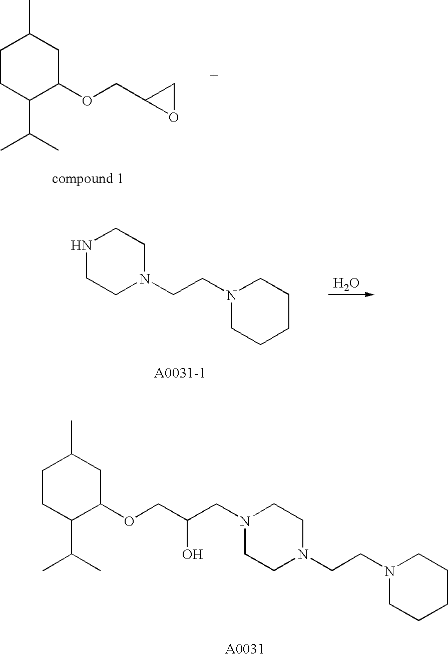

A mixture of A0031-1 (100 mg, 0.508 mmol), compound I (86 mg, 0.406 mmol) and H₂O (0.2 mL) was stirred overnight (about 18 hours) at room temperature. TLC showed that some of the start materials remained. The reaction mixture was then heated to 40° C. and stirred overnight (about 18 hours).

Next, the reaction mixture was extracted with ethyl acetate, washed with water and brine, dried with anhydrous sodium sulfate and concentrated under vacuum to afford 70 mg of crude product as a yellow liquid (yield: 41.9 μl).

To the crude product, 0.5 ml of Et₂O—HCl was added and a white solid appeared. The white solid was washed with PE/EA=7/1 two times. The pH of a solution containing the solid was adjusted to pH=2 with Na₂CO₃, then extracted with DCM. The organic layers were combined, washed with water and brine, dried with anhydrous sodium sulfate and concentrated under vacuum to afford 67 mg of the purified product (yield 40.3, confirmed by H NMR & MS, purity 91.8% by HPLC).

### Preparation of Compound A0040

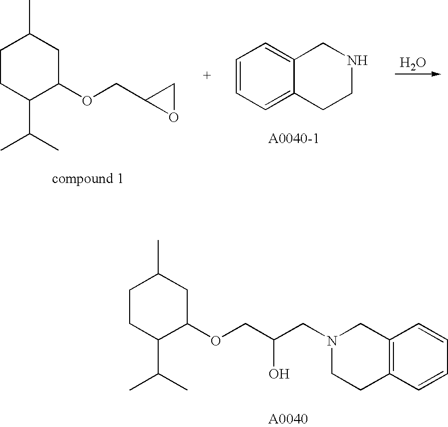

A mixture of compound A0040-1 (133 mg, 0.944 mmol), compound I (100 mg, 0.472 mmol) and H₂O (0.2 ml) was stirred overnight (about 18 hours) at room temperature. TLC showed that the reaction was complete. The reaction mixture was then extracted with ethyl acetate, washed with water and brine, dried with anhydrous sodium sulfate, and concentrated under vacuum to afford 221 mg of crude product. The crude product was purified via column chromatography (PE/EA=50/1 to PE/EA=10/1) to afford 100 mg of purified product (yield: 61.4%). The structure was confirmed by H NMR & MS, purity 94.6%; by HPLC.

### Preparation of Compound A0041-1

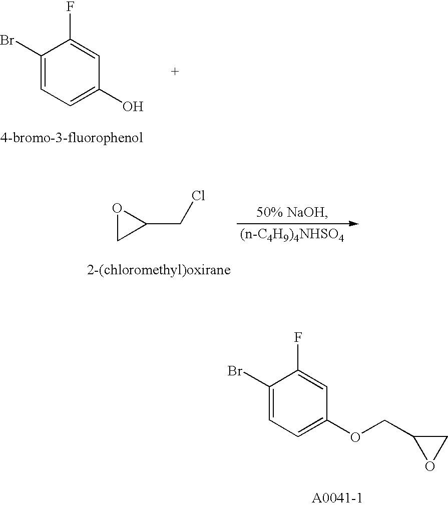

To a solution of NaOH (1.72 g, 43.1 mmol) in H₂O (2.3 ml) was added phenol (1 g, 5.2 mmol), (n-C₄H₉)₄N⁺HSO₄⁻ (70 mg, 0.208 mmol) and 2-(chloromethyl) oxirane (1.91 g, 20.8 mmol) at 0° C. The reaction mixture was then stirred overnight (about 18 hours) at room temperature. TLC showed that the reaction complete. Next, 30 ml of water was added to the reaction then extracted with CHCl₃. The organic layers were combined, washed with brine, dried with anhydrous sodium sulfate, and concentrated to provide the crude product. The crude product was purified via column chromatography (PE/CH₂Cl₂=2/1) to provide 700 mg of purified product (yield: 54%, confirmed by 1H NMR).

### Preparation of Compound A0041

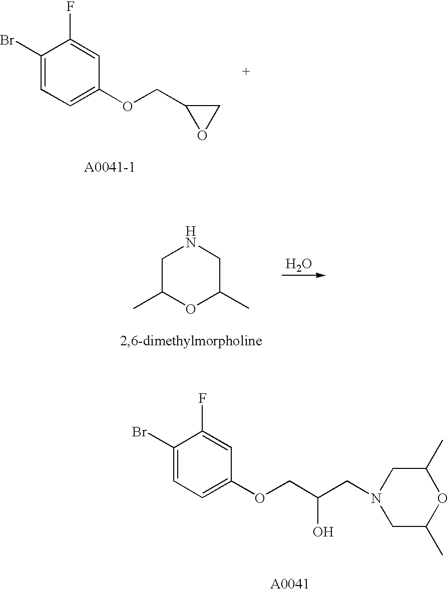

A mixture of 2,6-dimethylmorpholine (200 mg, 1.7 mmol), compound A0041-1 (210 mg, 0.85 mmol) and H₂O (0.2 ml) was stirred overnight (about 18 hours) at room temperature. TLC showed the reaction complete. The reaction mixture was then extracted with CHCl₃, washed with water and brine, dried with anhydrous sodium sulfate, and concentrated under vacuum to afford the crude product. The crude product was then purified via column chromatography (PE/EA=20/1 to PE/EA=1/1) to afford 132 mg of purified product (yield: 42.7%) confirmed by H NMR & LC-MS, purity 97.2% by HPLC).

### Preparation of Compound A0042-1

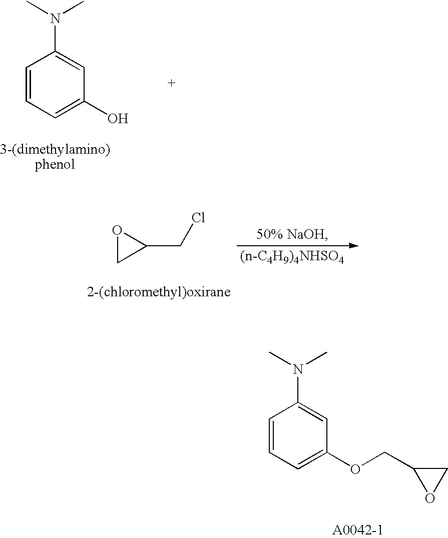

Run 1 (for A0050): To a solution of NaOH (1.21 g, 30.29 mmol) in H₂O (1.5 ml) was added phenol (500 mg, 3.65 mmol), (n-C₄H₉)₄N⁺HSO₄⁻ (50 mg, 0.146 mmol) and 2(chloromethyl)oxirane (1.35 g, 14.60 mmol) at 0° C. The reaction mixture was then stirred overnight (about 18 hours) at room temperature. TLC suggested the reaction complete. Next, 10 ml of water was added to the reaction mixture, then extracted with EA. The organic layers were combined, washed with brine, dried with anhydrous sodium sulfate, and concentrated to afford 320 mg of crude product. The crude product was then purified via column chromatography (PE/EA=50/1 to PE/EA=10/1) to afford 180 mg of the purified product (yield: 25.5%, confirmed by H NMR.

Run 2 (for A0042): To 1.2 ml of NaOH (50%) was added phenol (500 mg, 3.65 mmol), (n-C₄H₉)₄NHSO₄ (50 mg, 0.146 mmol) and 2-(chloromethyl)oxirane (1.14 ml, 14.6 mmol) at 0° C. The reaction mixture was then stirred overnight (about 18 hours) at room temperature. Next, 1N NaOH was added and the solution was extracted with DCM. The organic layers were combined, dried with anhydrous Na₂SO₄, and concentrated under vacuum. The crude product was then purified via column chromatography to obtain 160 mg of purified product as a colorless oil (yield: 22.7%.

### Preparation of Compound A0042

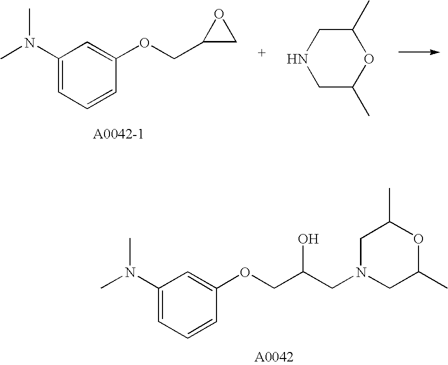

A mixture of A0042-1 (160 mg, 0.83 mmol), 2,6-dimethylmorpholine (0.2 ml, 1.66 mmol) and H₂O (0.3 mL) was stirred overnight (about 18 hours) at room temperature. TLC showed that some of the starting materials remained. The reaction mixture was then extracted with ethyl acetate, washed with water and brine, dried with anhydrous sodium sulfate, and concentrated under vacuum to afford 220 mg of crude product as yellow oil. The crude product was purified via column chromatography to obtain 100 mg of the purified product as yellow oil (yield: 39.2%, confirmed by H NMR, purity 97.4% by HPLC).

### Preparation of Compound A0043-1

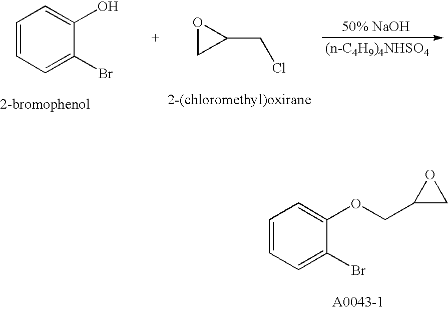

To 1.5 ml of NaOH (50%) was added 2-bromophenol (850 mg, 4.91 mmol), (n-C₄H₉)₄NHSO₄ (66 mg, 0.1965 mmol) and 2-(chloromethyl)oxirane (1.5 ml, 19.65 mmol) at 0° C. The reaction mixture was then stirred at room temperature overnight (about 18 hours). Next, 1N NaOH was added and the solution was extracted with DCM, the organic layers were combined, dried with anhydrous Na₂SO₄, and concentrated under vacuum. The crude product was purified via column chromatography to obtain 400 mg of purified product as a colorless oil (yield: 35.7%, confirmed by H NMR).

### Preparation of Compound A0044-1

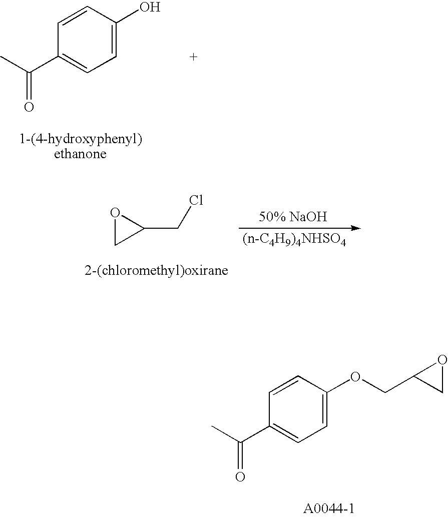

To a solution of 1-(4-hydroxyphenyl)-ethanone (500 mg, 3.67 mmol), (n-C₄H₉)₄N⁺HSO₄⁻ (50 mg, 0.147 mmol) and 2-(chloromethyl)oxirane (0.87 mL, 11 mmol) was added a solution of NaOH (1.2 g, 30.46 mmol) in H₂O (1.2 mL) at 0° C. The reaction mixture was then stirred overnight (about 18 hours) at room temperature. The reaction mixture was then poured into water (50 mL), extracted with DCM, and the organic layers were combined, dried with anhydrous sodium sulfate, and concentrated under vacuum to afford 1 g of crude product as a red liquid. The crude product was purified by silica gel to obtain 350 mg of purified product as a colorless liquid (yield: 49%, confirmed by H NMR).

### Preparation of Compound A0044

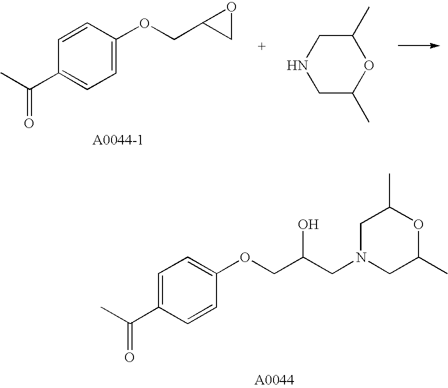

A mixture of 2,6-dimethylmorpholine (0.44 mL, 3.64 mmol), A0044-1 (350 mg, 1.82 mmol) and H₂O (0.6 mL) was stirred at room temperature overnight (about 18 hours). TLC suggested that the reaction was complete. The reaction mixture was then extracted with DCM, washed with brine, dried with anhydrous sodium sulfate, and concentrated under vacuum to the crude product as a yellow liquid. The crude product was then purified by silica gel to obtain 200 mg of the purified product as a white solid and 130 mg of the isomer as yellow liquid (yield: 35%, confirmed by H NMR & MS, purity 98.4% by HPLC).

### Preparation of Compound A0045-1

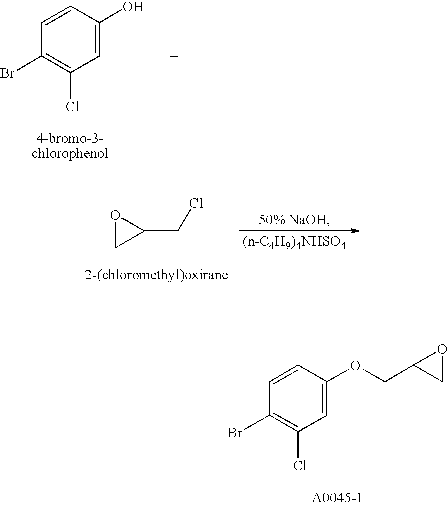

To a solution of 4-bromo-3-chlorophenol (500 mg, 2.41 mmol), (n-C₄H₉)₄N⁺HSO₄⁻ (33 mg, 0.0964 mmol) and 2-(chloromethyl)oxirane (0.57 mL, 7.23 mmol) was added a solution of NaOH (0.8 g, 20 mmol) in H₂O (0.8 mL) at 0° C. The reaction mixture was then stirred at room temperature overnight (about 18 hours). The reaction mixture was then poured into water (50 mL), extracted with DCM, and the organic layers were combined. Next, the organic layers were dried with anhydrous sodium sulfate and concentrated under vacuum to afford 0.6 g of crude product as a yellow liquid. The crude product was then purified by silica gel to obtain 280 mg of the purified product as a colorless oil (yield: 44%, confirmed by H NMR).

### Preparation of Compound A0046-1

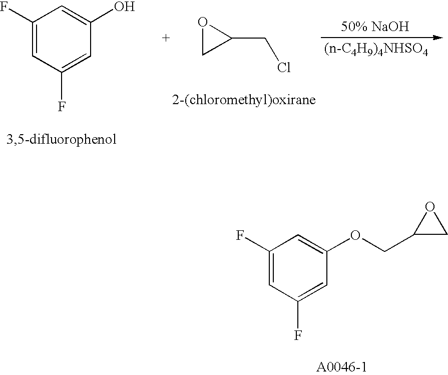

To 1.3 ml of NaOH (50%) was added 3,5-difluorophenol (0.528 g, 4.0 mmol), (n-C₄H₉)₄N⁺HSO₄⁻ (54 mg, 0.16 mmol) and 2-(chloromethyl)oxirane (1.48 g, 16 mmol) at 0° C. The reaction mixture was stirred overnight (about 18 hours) at room temperature. Next, 1N NaOH was added and the solution was extracted with DCM. The organic layers were combined, dried with anhydrous Na₂SO₄, and concentrated under vacuum. The crude product was purified via column chromatography to obtain 300 mg of the purified product as colorless oil (yield: 40%, confirmed by H NMR).

### Preparation of Compound A0046

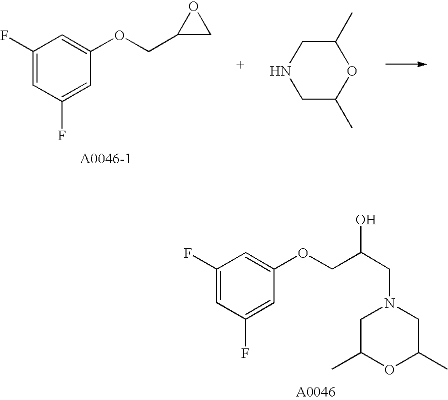

A mixture of A0046-1 (156 mg, 0.83 mmol), 2,6-dimethylmorpholine (191 mg, 1.66 mmol) and H₂O (0.2 mL) was stirred overnight (about 18 hours) at room temperature. The reaction mixture was then extracted with ethyl acetate, washed with water and brine, dried with anhydrous sodium sulfate, and concentrated under vacuum. The crude product was purified via column chromatography to obtain the purified product (132 mg) as a colorless oil (yield: 52.8%). The structure was confirmed by H NMR & MS, purity 97.4% by HPLC.

### Preparation of Compound A0047-1

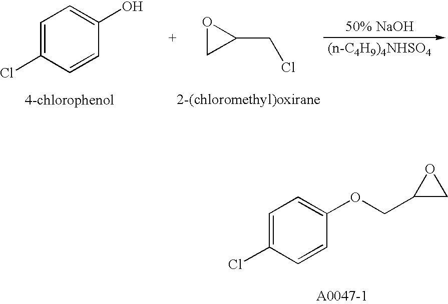

To 1.3 ml of NaOH (50%) was added 4-chlorophenol (500 mg, 3.9 mmol), (n-C₄H₉)₄N⁺HSO₄⁻ (53 mg, 0.156 mmol) and 2-(chloromethyl)oxirane (902 mg, 9.75 mmol) at 0° C. The reaction mixture was then stirred at room temperature overnight (about 18 hours). Next, 1N NaOH was added and the solution was extracted with DCM, the organic layers were combined, dried with anhydrous Na₂SO₄ and concentrated under vacuum. The crude product was purified via column chromatography to obtain 370 mg of the purified product as a yellow oil (yield: 51.5%, confirmed by H NMR).

### Preparation of Compound A0047

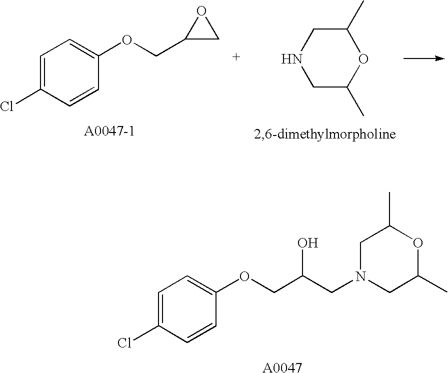

A mixture of A0047-1 (332 mg, 1.8 mmol), 2,6-dimethylmorpholine (0.44 ml, 3.6 mmol) and H₂O (0.6 mL) was stirred at room temperature overnight (about 18 hours). TLC showed that some of the starting materials remained. The reaction mixture was extracted with ethyl acetate, washed with water and brine, dried with anhydrous sodium sulfate and concentrated under vacuum to afford 520 mg of crude product as yellow oil (yield: 96.6%).

About 120 mg of the crude product was purified by Pre-TLC to obtain 62 mg of the purified product (yield: 51.6%, confirmed by H NMR & MS, purity 98.4% by HPLC).

### Preparation of Compound A0049

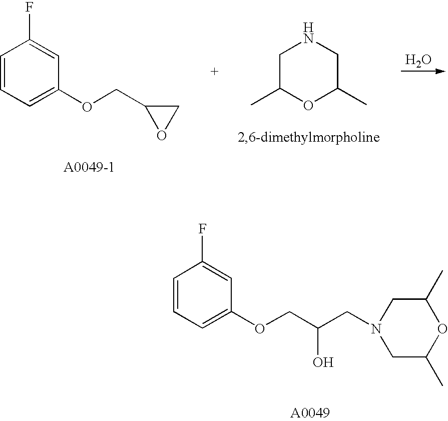

A mixture of 2,6-dimethylmorpholine (137 mg, 1.190 mmol), compound A0049-1 (100 mg, 0.595 mmol) and H₂O (0.2 ml) was stirred overnight at room temperature. TLC suggested that the reaction complete. The reaction mixture was then extracted with ethyl acetate, washed with water and brine, dried with anhydrous sodium sulfate, and concentrated under vacuum to afford 220 mg of the crude product. The crude product was purified via column chromatography (PE/EA=50/1 to PE/EA=10/1) to afford 101 mg of purified product (yield: 59.9%, confirmed by H NMR, purity 96.8% by HPLC).

### Preparation of Compound A0049-1

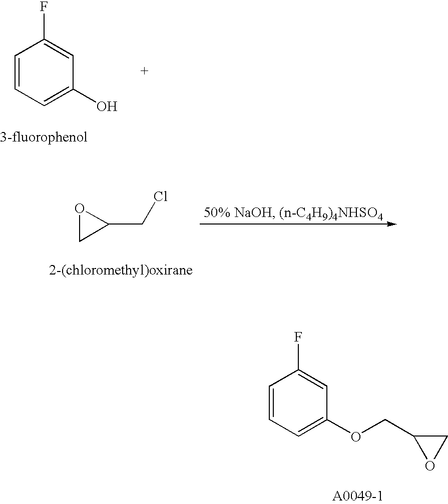

To a solution of NaOH (1.480 g, 37.01 mmol) in H₂O (2 ml) was added phenol (500 mg, 4.46 mmol), (n-C₄H₉)₄N⁺HSO₄⁻ (60 mg, 0.178 mmol) and 2-(chloromethyl)oxirane (1.659 g, 17.85 mmol) 0° C. The reaction mixture was then stirred overnight at room temperature. TLC suggested that the reaction was complete. Next, 10 ml of water was added to the reaction mixture, which was then extracted with EA. The organic layers were combined, washed with brine, dried with anhydrous sodium sulfate, and concentrated to afford 641 mg of crude product. The crude product was purified via column chromatography (PE/EA=100/1 to PE/EA=50/1) to afford 421 mg of purified product (yield: 56.2%). The structure was confirmed by H NMR, shown as follows.

Each of the patents, patent applications and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed:

1. A compound of Formula I 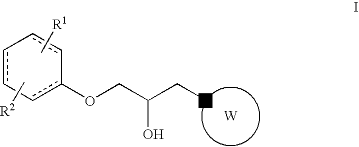
R¹ and R² are the same or different and are independently selected from the group consisting of H, halogen, C₁-C₆ hydrocarbyl, C₁-C₆ acyl, C₁-C₆ hydrocarbyloxy and NR³R⁴ wherein R³ and R⁴ are the same or different and are H, C₁-C₄ hydrocarbyl, C₁-C₄ acyl, C₁-C₄ hydrocarbylsulfonyl, or R³ and R⁴ together with the depicted nitrogen form a 5-7-membered ring that optionally contains 1 or 2 additional hetero atoms that independently are nitrogen, oxygen or sulfur;
W is a ring structure that contains 5 to 8 atoms in the ring including the depicted nitrogen, and can optionally contain
a) 1 or 2 further hetero atoms that are independently oxygen, nitrogen or sulfur, and
b) one or more substituent groups bonded to one or more ring atoms, said one or more substituent containing a total of up to 12 atoms selected from the group consisting of carbon, nitrogen, oxygen and sulfur, and mixtures thereof;
a dotted line ( [symbol] ) represents 1, 2, or 3 optional double bonds,
with the proviso that R¹ and R² are other than methyl and isopropyl, respectively, when W is N-morpholinyl or dimethyl-N-morpholinyl, and the three optional double bonds are absent.

2. The compound according to claim 1, wherein when one optional double bond is present, three double bonds are present.

3. The compound according to claim 2, wherein said three double bonds are present and one of R¹ and R² is H.

4. The compound according to claim 3, wherein one of R¹ and R² is C₁-C₄ acyl.

5. The compound according to claim 3, wherein one of R¹ and R² is NR³R⁴.

6. The compound according to claim 2, wherein said three double bonds are present and both of R¹ and R² are halogen.

7. A compound of Formula Ia 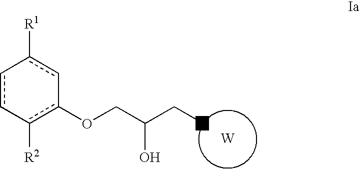
wherein
R¹ and R² are the same or different and are independently H, or C₁-C₆ hydrocarbyl;
W is a ring structure that contains 5 to 8 atoms in the ring including the depicted nitrogen, and can optionally contain
a) 1 or 2 further hetero atoms that are independently oxygen, nitrogen or sulfur, and
b) one or more substituent groups bonded to one or more ring atoms, said one or more substituent containing a total of up to 12 atoms selected from the group consisting of carbon, nitrogen, oxygen and sulfur, and mixtures thereof;
a dotted line ( [symbol] ) represents 1, 2, or 3 optional double bonds,
with the proviso that R¹ and R² are other than methyl and isopropyl, respectively, when W is N-morpholinyl or dimethyl-N-morpholinyl and the three optional double bonds are absent.

8. The compound according to claim 7, wherein W contains at least one additional hetero atom.

9. The compound according to claim 8, wherein W includes one or more substituent groups bonded to one or more ring atoms, said one or more substituent containing a total of up to 12 atoms selected from the group consisting of carbon, nitrogen, oxygen and sulfur, and mixtures thereof.

10. The method according to claim 7, wherein W is selected from the group consisting of

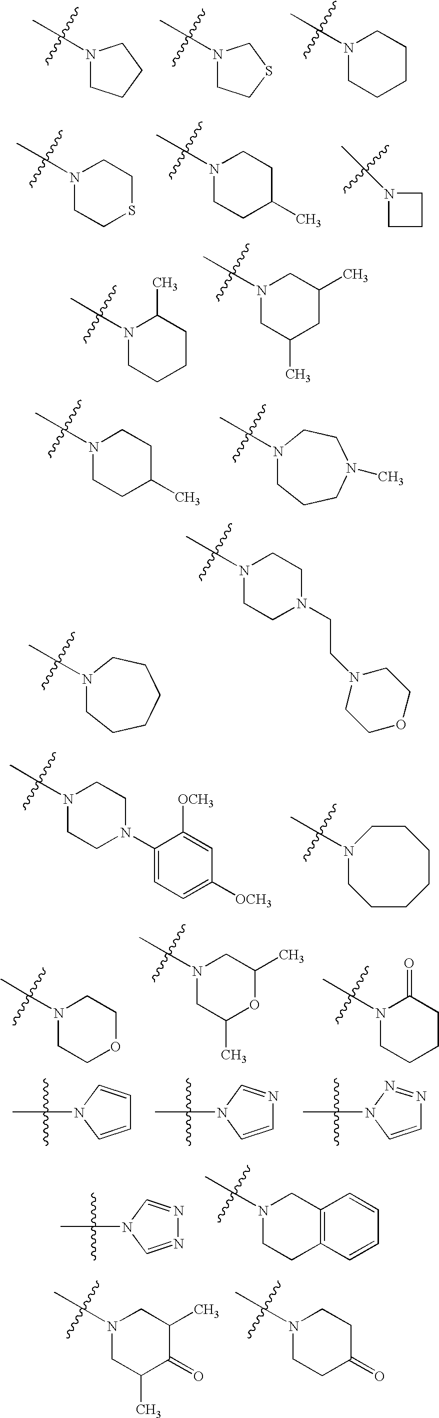

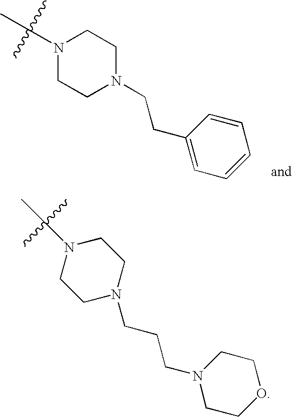

11. The compound according to claim 7, wherein said compound of Formula I has the structure of Formula II 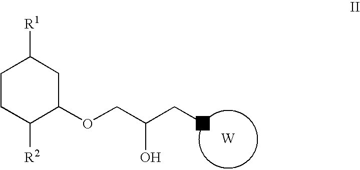
wherein R¹ and R² and W are as previously defined.

12. The compound according to claim 11, wherein R¹ is methyl and R² contains 3 to 5 carbon atoms.

13. The compound according to claim 11 that is selected from the group consisting of

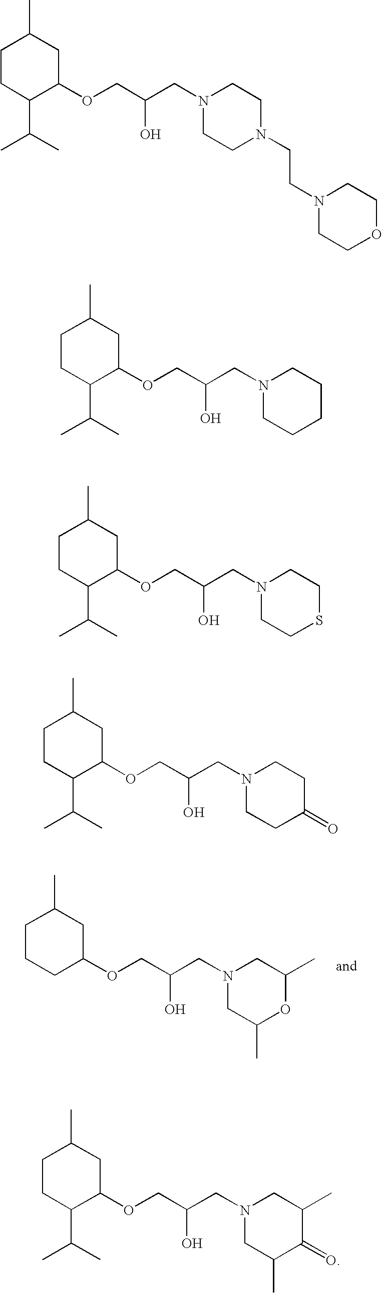

14. The compound according to claim 7, wherein said compound of Formula I has the structure of Formula III 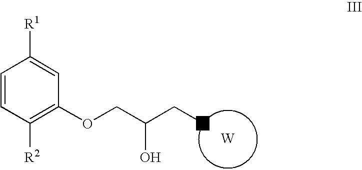
wherein R¹ and R² and W are as previously defined.

15. The compound according to claim 14, wherein one of R¹ and R² is H, and the other contains 3 to 5 carbon atoms.

16. The compound according to claim 14 that is selected from the group consisting of

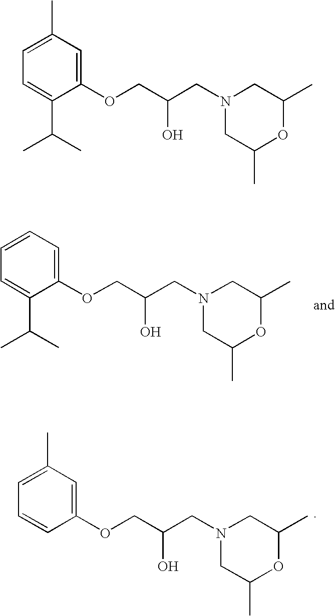

17. A pharmaceutical composition comprising an analgesic effective amount of a compound of Formula I dissolved or dispersed in a physiologically tolerable carrier 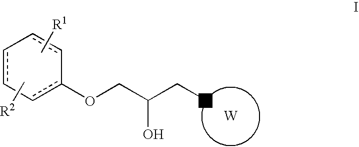
wherein
R¹ and R² are the same or different and are independently selected from the group consisting of H, halogen, C₁-C₆ hydrocarbyl, C₁-C₆ acyl, C₁-C₆ hydrocarbyloxy and NR³R⁴ wherein R³ and R⁴ are the same or different and are H, C₁-C₄ hydrocarbyl, C₁-C₄ acyl, C₁-C₄ hydrocarbylsulfonyl, or R³ and R⁴ together with the depicted nitrogen form a 5-7-membered ring that optionally contains 1 or 2 additional hetero atoms that independently are nitrogen, oxygen or sulfur;
W is a ring structure that contains 5 to 8 atoms in the ring including the depicted nitrogen, and can optionally contain
a) 1 or 2 further hetero atoms that are independently oxygen, nitrogen or sulfur, and
b) one or more substituent groups bonded to one or more ring atoms, said one or more substituent containing a total of up to 12 atoms selected from the group consisting of carbon, nitrogen, oxygen and sulfur, and mixtures thereof;
a dotted line ( [symbol] ) represents 1, 2, or 3 optional double bonds,
with the proviso that R¹ and R² are other than methyl and isopropyl, respectively, when W is N-morpholinyl or dimethyl-N-morpholinyl, and the three optional double bonds are absent.

18. The pharmaceutical composition according to claim 17, wherein said compound of Formula I has the structure of Formula Ia 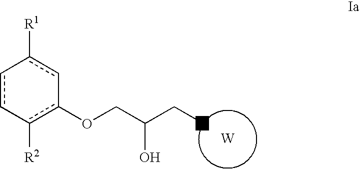
wherein
R¹ and R² are the same or different and are independently H, or C₁-C₆ hydrocarbyl;
W is a ring structure that contains 5 to 8 atoms in the ring including the depicted nitrogen, and can optionally contain
a) 1 or 2 further hetero atoms that are independently oxygen, nitrogen or sulfur, and
b) one or more substituent groups bonded to one or more ring atoms, said one or more substituent containing a total of up to 12 atoms selected from the group consisting of carbon, nitrogen, oxygen and sulfur, and mixtures thereof;
a dotted line ( [symbol] ) represents 1, 2, or 3 optional double bonds,
with the proviso that R¹ and R² are other than methyl and isopropyl, respectively, when W is N-morpholinyl or dimethyl-N-morpholinyl, and the three optional double bonds are absent.

19. The pharmaceutical composition according to claim 17, wherein said compound of Formula I has the structure of Formula II

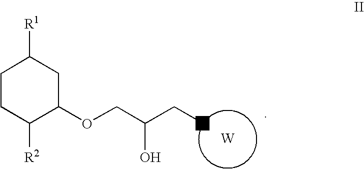

20. The pharmaceutical composition according to claim 17 wherein said compound of Formula I has the structure of Formula III

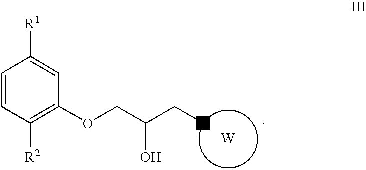

21. A method of reducing pain in a host mammal in need thereof that comprises administering to that host mammal a pharmaceutical composition containing an analgesic effective amount of a compound of Formula I dissolved or dispersed in a physiologically tolerable carrier 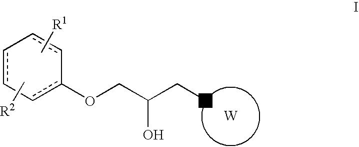
wherein
R¹ and R² are the same or different and are independently selected from the group consisting of H, halogen, C₁-C₆ hydrocarbyl, C₁-C₆ acyl, C₁-C₆ hydrocarbyloxy and NR³R⁴ wherein R³ and R⁴ are the same or different and are H, C₁-C₄ hydrocarbyl, C₁-C₄ acyl, C₁-C₄ hydrocarbylsulfonyl, or R³ and R⁴ together with the depicted nitrogen form a 5-7-membered ring that optionally contains 1 or 2 additional hetero atoms that independently are nitrogen, oxygen or sulfur;
W is a ring structure that contains 5 to 8 atoms in the ring including the depicted nitrogen, and can optionally contain
a) 1 or 2 further hetero atoms that are independently oxygen, nitrogen or sulfur, and
b) one or more substituent groups bonded to one or more ring atoms, said one or more substituent containing a total of up to 12 atoms selected from the group consisting of carbon, nitrogen, oxygen and sulfur, and mixtures thereof;
a dotted line ( [symbol] ) represents 1, 2, or 3 optional double bonds.

22. The method according to claim 21, wherein said compound of Formula I is a compound of Formula Ia 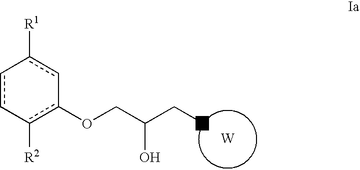
wherein
R¹ and R² are the same or different and are independently H, or C₁-C₆ hydrocarbyl;
W is a ring structure that contains 5 to 8 atoms in the ring including the depicted nitrogen, and can optionally contain 1 or 2 further hetero atoms that are independently oxygen, nitrogen or sulfur; and
a dotted line ( [symbol] ) represents 1, 2, or 3 optional double bonds.

23. The method according to claim 22, wherein W contains at least one additional hetero atom.

24. The method according to claim 22, wherein W further includes one or more substituent groups bonded to one or more ring atoms, said one or more substituent containing a total of up to 12 atoms selected from the group consisting of carbon, nitrogen, oxygen and sulfur, and mixtures thereof.

25. The method according to claim 22, wherein W is selected from the group consisting of

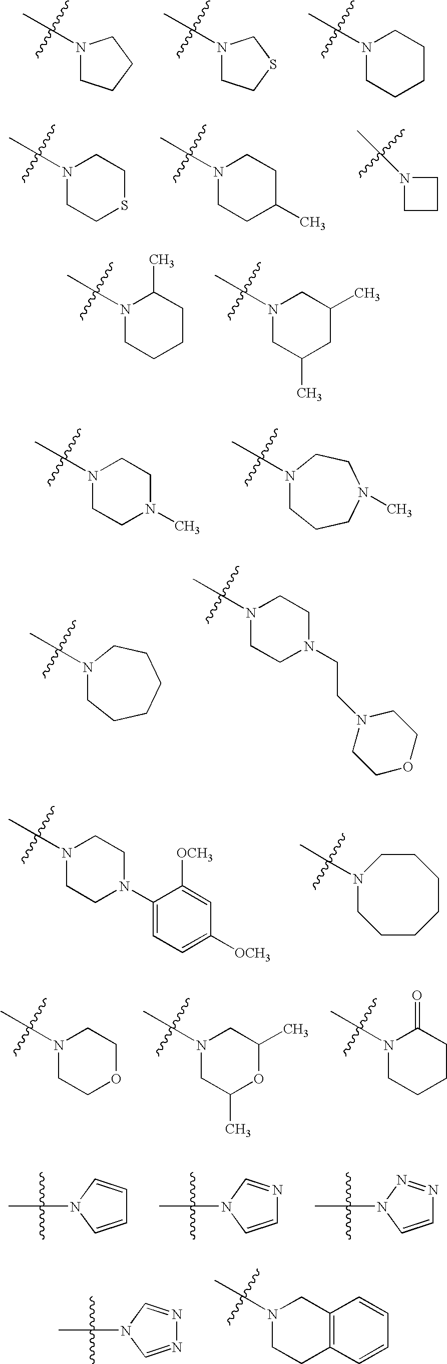

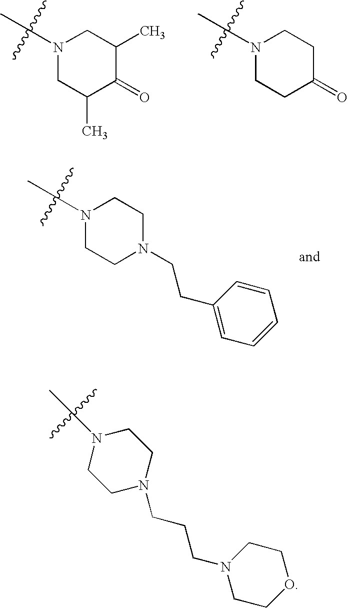

26. The method according to claim 22, wherein said host mammal is selected from the group consisting of a primate, a laboratory rodent, a companion animal, and a food animal.

27. The method according to claim 22, wherein said composition is administered a plurality of times over a period of days.

28. The method according to claim 27, wherein said composition is administered a plurality of times in one day.

26. The method according to claim 22, wherein said compound of Formula Ia has the structure of Formula II 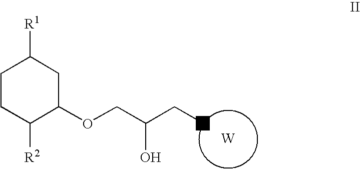
wherein R¹ and R² and W are as previously defined.

27. The method according to claim 26, wherein R¹ is methyl and R² contains 3 to 5 carbon atoms.

28. The method according to claim 26 wherein said compound of Formula II is selected from the group consisting of

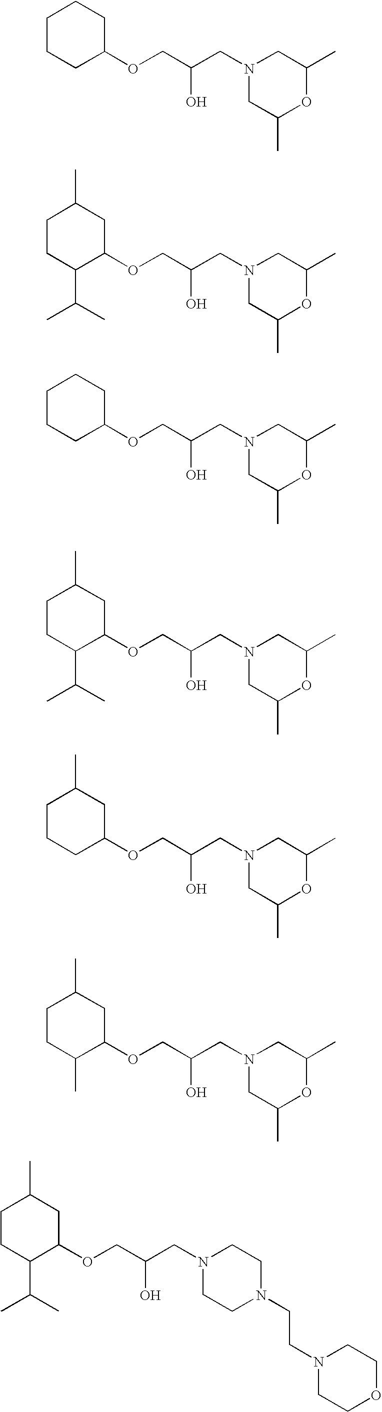

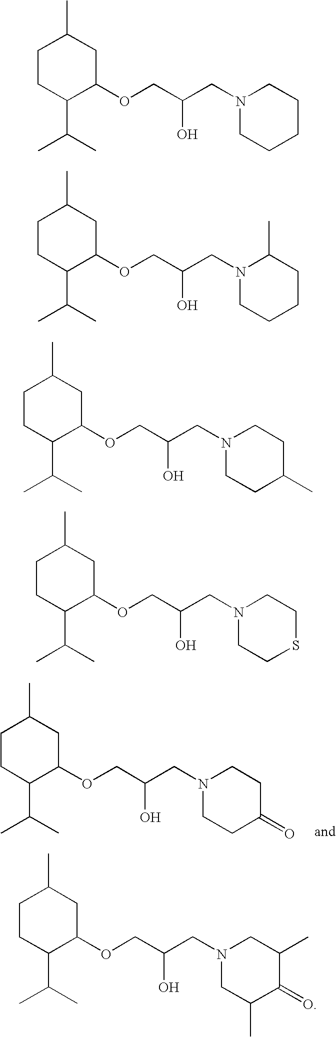

29. The method according to claim 22, wherein said compound of Formula Ia has the structure of Formula III 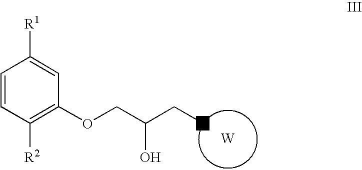
wherein R¹ and R² and W are as previously defined.

30. The method according to claim 29, wherein one of R¹ and R² is H, and the other contains 3 to 5 carbon atoms.

31. The method according to claim 29 wherein said compound of Formula III is selected from the group consisting of [chemical structure]


SEQUENCE LISTING (1 sequence)

SEQ ID NO: 1 - protein, 5 aa, Artificial sequence
  chemically synthesized FLNA sequence that corresponds to amino acid residue positions 2561-2565 of the FLNA protein
VAKGL